(19) United States
(12) Patent Application Publication
(10) Pub. No.: US 2023/0364007 A1
(43) Pub. Date: Nov. 16, 2023

(54) ADMINISTRATION OF A PSYCHEDELIC COMPOUND

(71) Applicant: Reset Pharmaceuticals, Inc., New York, NY (US)

(72) Inventors: Harri DICKINSON, Cheadle (GB); Paul Alfred DICKINSON, Cheadle (GB); Nicola PARISI, Cheadle (GB)

(21) Appl. No.: 18/196,567

(22) Filed: May 12, 2023

Related U.S. Application Data
(60) Provisional application No. 63/364,633, filed on May 13, 2022.
(60) Provisional application No. 63/364,632, filed on May 13, 2022.
(60) Provisional application No. 63/364,634, filed on May 13, 2022.

(30) Foreign Application Priority Data
May 26, 2022 (GB) 2207765.5
May 26, 2022 (GB) 2207766.3
May 26, 2022 (GB) 2207767.1

(51) Int. Cl. A61K 9/00; A61K 31/675; A61K 31/4045; A61K 9/10; A61K 47/32; A61K 47/10; A61K 47/40; A61K 47/44

(52) U.S. Cl. CPC A61K 9/0019; A61K 31/675; A61K 31/4045; A61K 9/10; A61K 47/32; A61K 47/10; A61K 47/40; A61K 47/44

(57) ABSTRACT

The invention relates to a method of treating or preventing a disease or condition comprising administering a therapeutically effective amount of a psychedelic compound to a patient by subcutaneous injection. Uses, formulations and kits are also described.

### FIELD OF THE INVENTION

The present invention relates to methods of treating or preventing a disease or condition, comprising administering a psychedelic compound to a patient. Kits and formulations are also described.

### BACKGROUND OF THE INVENTION

Psilocybin (4-phosphoryloxy-N,N-dimethyltryptamine) is a tryptamine serotoninergic psychedelic. The IUPAC name of psilocybin is [3-(2-dimethylaminoethyl)-1H-indol-4-yl] dihydrogen phosphate. The structure of psilocybin is shown below.

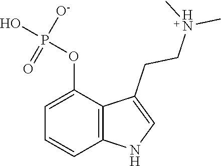

Psilocybin is metabolized in the body to psilocin (4-hydroxy-N,N-dimethyltryptamine), which exerts its effects primarily via 5HT2A agonism. The structure of psilocin is shown below.

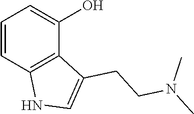

Psilocybin and psilocin are currently being investigated as a potential treatment for various psychological, neurological and central nervous system disorders (for example, demoralization, depression, anxiety and adjustment disorders) in a variety of clinical settings.

Psilocybin is typically dosed orally. Following oral dosing, the onset of action typically starts between 20-40 minutes, with maximum effect at 60-90 minutes and a duration of 4-8 hours. A faster onset of action and a shorter duration of the psychological effect than those reported above for oral administration would be desirable when psilocybin or psilocin are used as therapeutic agents. Furthermore, the bioavailability of psilocybin has been reported to be approximately 50%. An improvement of the bioavailability would allow a desirable reduction of the psilocybin dose needed to elicit its action.

Intravenous (IV) administration of psilocybin has been reported to have an onset of action of 1-2 minutes, and a duration of the psychological effects of 20 minutes. A slower onset of action and a longer duration of the psychological effects than those previously observed for IV administration need to be achieved for the administration of psilocybin or psilocin to be therapeutically useful, for example for treatment of psychiatric disorders. An improved control of the pharmacokinetic (PK) profile would allow optimization of the dose of psilocybin or psilocin and reduction of PK variability improving the effectiveness of treatment and reducing adverse effects.

There is accordingly a need to develop new methods of administration of psilocybin and psilocin which allow improvements to the control of the pharmacokinetic profile to be obtained. Such new methods would provide improved treatments, for example when psilocybin or psilocin are used in the treatment of psychiatric disorders.

### SUMMARY OF THE INVENTION

It is a finding of the invention that a subcutaneous (SC) injection dosage form of psilocybin or psilocin provides improved methods of treatment or prevention of diseases or conditions. In particular, controlling the formulation type and dose administered for a SC injection dosage form of psilocybin or psilocin allows a desirable onset of action, duration of psychological effects and pharmacokinetic (PK) profile to be achieved when the psilocybin or psilocin is administered to a patient.

The present invention accordingly provides a method of treating or preventing a disease or condition in a patient, the method comprising administering a therapeutically effective amount of a psychedelic compound to the patient by subcutaneous injection, wherein the psychedelic compound is psilocybin or psilocin, or a pharmaceutically acceptable salt thereof.

The invention further provides a psychedelic compound for use in a method of treating or preventing a disease or condition in a patient, wherein: the psychedelic compound is psilocybin or psilocin, or a pharmaceutically acceptable salt thereof; and the method comprises administering a therapeutically effective amount of the psychedelic compound to the patient by subcutaneous injection.

The invention also provides use of a psychedelic compound in the manufacture of a medicament for use in a method of treating or preventing of a disease or condition in a patient, wherein: the psychedelic compound is psilocybin or psilocin, or a pharmaceutically acceptable salt thereof; and the method comprises administering a therapeutically effective amount of the psychedelic compound to the patient by subcutaneous injection.

The disease or condition may be selected from psychological, neurological and central nervous system disorders.

The invention further provides a formulation suitable for administration by subcutaneous injection comprising a psychedelic compound which is psilocybin or a pharmaceutically acceptable salt thereof, wherein the formulation is a suspension and the formulation comprises the psychedelic compound at a concentration of at least about 70 mg/g.

The invention further provides a formulation suitable for administration by subcutaneous injection comprising a psychedelic compound which is psilocin or a pharmaceutically acceptable salt thereof, wherein the formulation is a suspension or a solution and the formulation comprises psilocin at a concentration of at least about 200 mg/g.

Also provided by the invention is a kit comprising: one or more formulations suitable for administration by subcutaneous injection, which one or more formulations comprise a psychedelic compound which is psilocybin or psilocin, or a pharmaceutically acceptable salt thereof; and instructions for use of the one or more formulations in a method as defined herein.

### BRIEF DESCRIPTION OF THE FIGURES

FIG. 1 shows a model schematic of a two-compartment IV bolus model.

FIG. 2 shows visualized plasma concentration data.

FIG. 3 shows a model schematic of a SC one-compartment model.

FIG. 4 shows psilocin blood concentration following subcutaneous administration of psilocybin to rats at 4 dose levels: 0.05 mg/kg, 0.1 mg/kg, 1 mg/kg and 10 mg/kg.

FIG. 5 shows a fitted one compartment SC PK model of psilocin in rats where the lines are simulated PK profile and dots are the SC data.

FIG. 6 shows a model schematic of a two-compartment model with one absorption rate for SC administration.

FIG. 7 shows human simulations of psilocin exposure after SC administration over a range of doses, where the plot is faceted by dose in mg.

FIG. 8 shows human simulations of psilocin exposure after SC administration with slower absorption rate (44.8 minute absorption half-life) over a range of doses, where the plot is faceted by dose in mg.

### DETAILED DESCRIPTION OF THE INVENTION

The method of the invention comprises administering a therapeutically effective amount of a psychedelic compound to the patient by subcutaneous injection. In the present invention, the psychedelic compound is psilocybin or psilocin, or a pharmaceutically acceptable salt thereof. In one embodiment, the psychedelic compound is psilocybin or a pharmaceutically acceptable salt thereof. In one embodiment, the psychedelic compound is psilocin or a pharmaceutically acceptable salt thereof.

As used herein, the term “subcutaneous injection” or “SC injection” means administration of a substance by injection directly into a subject's subcutaneous tissue. For example, the injection may be to the outer area of the upper arm, the abdomen, the front of the thigh, the upper back, the buttock, or to any other area suitable for subcutaneous injection.

As used herein, the term “intravenous infusion” or “IV infusion” means administration of a substance directly into a subject's veins.

The psychedelic compound administered to the patient may be in any pharmaceutically acceptable form. For instance, the psychedelic compound may be in the form of a solvate, a hydrate, a crystal, or a co-crystal. Typically, the psychedelic compound is administered to the patient in the form of a solution or suspension comprising the psychedelic compound. The psychedelic compound may be administered to the patient in the form of a solution comprising the psychedelic compound. Alternatively, the psychedelic compound may be administered to the patient in the form of a suspension comprising the psychedelic compound. The psychedelic compound may be formulated as one of the formulation types discussed herein.

The psychedelic compound may be administered to the patient by any suitable method for subcutaneous injection. For example, the psychedelic compound may be administered to the outer area of the upper arm, the abdomen, the front of the thigh, the upper back, the buttock, or to any other area suitable for subcutaneous injection.

The therapeutically effective amount of the psychedelic compound may be from about 0.5 mg to about 25 mg, from about 1 mg to about 24 mg, from about 1.5 mg to about 23 mg, from about 2 mg to about 22 mg, from about 2.5 mg to about 21 mg, from about 3 mg to about 20 mg, from about 3.5 mg to about 19 mg, from about 4 mg to about 18 mg, from about 4.5 mg to about 17 mg, from about 5 mg to about 16 mg, from about 5.5 mg to about 15 mg, from about 6 mg to about 14 mg, from about 6.5 mg to about 13 mg, or from about 7 mg to about 12 mg. In one embodiment, the psychedelic compound is psilocybin and the therapeutically effective amount is from about 6 mg to about 14 mg, or from about 7 mg to about 12 mg. In another embodiment, the psychedelic compound is psilocin and the therapeutically effective amount is from about 4.5 mg to about 10 mg, or from about 5 mg to about 8.5 mg.

As used herein, the term “about” means any value that the skilled person would appreciate is a reasonable variation of the value that is referred to by the term “about”. Typically, “about” means±10% or ±5%.

For example, the therapeutically effective amount of the psychedelic compound may be from about 1.5 mg to about 2.5 mg, from about 2.5 mg to about 3.5 mg, from about 3.5 mg to about 4.5 mg, from about 4.5 mg to about 5.5 mg, from about 5.5 mg to about 6.5 mg, from about 6.5 mg to about 7.5 mg, from about 7.5 mg to about 8.5 mg, from about 8.5 mg to about 9.5 mg, from about 9.5 mg to about 10.5 mg, from about 10.5 mg to about 11.5 mg, from about 11.5 mg to about 12.5 mg, from about 12.5 mg to about 13.5 mg, from about 13.5 mg to about 14.5 mg, from about 14.5 mg to about 15.5 mg, from about 15.5 mg to about 16.5 mg, or from about 16.5 mg to about 17.5 mg.

As discussed above, an SC injection dosage form of the psychedelic compound allows a desirable onset of action, and duration of psychological effects to be obtained. In particular, the method of the present invention allows a tuneable PK profile to be obtained by controlling the dose of the injection and/or rate of absorption of the psychedelic compound by a patient.

As used herein, the term “duration of psychological effects” refers to the total time that a therapeutic effect of the compound is observed for. This may be determined by measuring psilocin levels in the patient's blood plasma. In that case, psychological effects may be observed when the blood plasma concentration of psilocin is above about 5 ng/mL. Alternatively, the onset and end of the psychological effects may be based on observation by a medical professional, or they may be self-reported by the patient.

A finding of the present invention is that an advantageous maximum blood plasma concentration (Cₘₐₓ) of psilocin (i.e. the psychedelic compound itself when psilocin is administered, or the active metabolite when psilocybin is administered) may be obtained by administering the psychedelic compound to a patient by subcutaneous injection as discussed herein. Such advantageous Cₘₐₓ value of psilocin provides a desirable therapeutic effect, thus providing effective treatment or prevention of the diseases, disorders and conditions described herein, without causing an excessive duration of the psychological effect in the patient.

For example, administering the therapeutically effective amount of the psychedelic compound to the patient by subcutaneous injection may achieve a maximum blood plasma concentration (Cₘₐₓ) of psilocin in the patient which is at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, or at least about 15 ng/mL.

For example, administering the therapeutically effective amount of the psychedelic compound to the patient by subcutaneous injection may achieve a maximum blood plasma concentration (Cₘₐₓ) of psilocin in the patient which is no greater than about 20 ng/mL, no greater than about 19 ng/mL, no greater than about 18 ng/mL, no greater than about 17 ng/mL, no greater than about 16 ng/mL, no greater than about 15 ng/mL, no greater than about 14 ng/mL, no greater than about 13 ng/mL, no greater than about 12 ng/mL, no greater than about 11 ng/mL, or no greater than about 10 ng/mL.

Thus, administering the therapeutically effective amount of the psychedelic compound to the patient by subcutaneous injection may achieve a maximum blood plasma concentration (Cₘₐₓ) of psilocin in the patient which is, for example, from about 3 ng/mL to about 4 ng/mL, from about 4 ng/mL to about 5 ng/mL, from about 5 ng/mL to about 6 ng/mL, from about 6 ng/mL to about 7 ng/mL, from about 7 ng/mL to about 8 ng/mL, from about 8 ng/mL to about 9 ng/mL, from about 9 ng/mL to about 10 ng/mL, from about 10 ng/mL to about 11 ng/mL, from about 11 ng/mL to about 12 ng/mL, from about 12 ng/mL to about 13 ng/mL, from about 13 ng/mL to about 14 ng/mL, from about 14 ng/mL to about 15 ng/mL, from about 15 ng/mL to about 16 ng/mL, from about 16 ng/mL to about 17 ng/mL, from about 17 ng/mL to about 18 ng/mL, from about 18 ng/mL to about 19 ng/mL, or from about 19 ng/mL to about 20 ng/mL.

In general, during the method of the invention it is desirable to achieve a blood plasma concentration of psilocin that is between about 5 ng/mL and about 10 ng/mL for an extended period of time over the course of the treatment. This range is preferred in particular when the method is used in the treatment of prevention of psychological, neurological and central nervous system disorders.

As discussed above, it is desirable for the method of the present invention to result in a faster onset of action and a shorter duration of the psychological effect than those reported for oral administration when psilocybin or psilocin are used as therapeutic agents.

It is generally desirable for the method of the invention to achieve onset of therapeutic action relatively quickly after the subcutaneous injection. Thus, administering the therapeutically effective amount of the psychedelic compound to the patient by subcutaneous injection may achieve onset of therapeutic action in a time from onset of subcutaneous injection of less than about 90 minutes, or less than about 80 minutes, or less than about 70 minutes, or less than about 60 minutes, or less than about 50 minutes, or less than about 40 minutes, or less than about 30 minutes, or less than about 20 minutes, or less than about 10 minutes, or less than about 5 minutes.

As used herein, the term “onset of therapeutic action” refers to the point during administration of the psychedelic compound where the therapeutic effect of the compound is first observed. This may be determined by measuring psilocin levels in the patient's blood plasma. In that case, the blood plasma concentration may be around about 5 ng/mL at the point of onset of therapeutic action. Alternatively, the onset of therapeutic action may be based on observation by a medical professional, or it may be self-reported by the patient during the administration of the compound.

It is also generally desirable for the onset of therapeutic action to not be too rapid after the subcutaneous injection. Thus, administering the therapeutically effective amount of the psychedelic compound to the patient by subcutaneous injection may achieve onset of therapeutic action in a time from onset of subcutaneous injection of greater than about 1 minutes, or greater than about 2 minutes, or greater than about 3 minutes, or greater than about 4 minutes, or greater than about 5 minutes, or greater than about 6 minutes, or greater than about 7 minutes.

Thus, onset of therapeutic action is typically from about 2 minutes to about 60 minutes, or from about 3 minutes to about 50 minutes, or from about 4 minutes to about 40 minutes, or from about 5 minutes to about 30 minutes, or from about 6 minutes to about 20 minutes after the subcutaneous injection.

Administering the therapeutically effective amount of the psychedelic compound to the patient by subcutaneous injection may achieve a blood plasma concentration of psilocin of greater than or equal to about 5 ng/mL in a time from the subcutaneous injection of from about 1 to about 60 minutes, or from about 2 to about 50 minutes, or from about 3 to about 40 minutes, or from about 4 to about 30 minutes, or from about 5 to about 20 minutes. Typically, administering the therapeutically effective amount of the psychedelic compound to the patient by subcutaneous injection achieves a blood plasma concentration of psilocin of greater than or equal to about 5 ng/mL in a time from the subcutaneous injection of from about 6 to about 15 minutes.

The method of the invention may achieve a blood plasma concentration of psilocin of greater than about 5 ng/mL for a relatively short period of time, in particular to provide a shorter duration of the psychological effect than can be obtained by oral administration of psilocybin or psilocin. This provides improvements when the method of the invention is used, for example, to treat disorders such as psychological, neurological and central nervous system disorders. In particular, the relatively short duration of psychological effects provided by the invention may reduce the duration of time that a patient must be supervised and/or monitored following therapy with a psychedelic agent, such as psilocybin or psilocin.

Thus, administering the therapeutically effective amount of the psychedelic compound to the patient by subcutaneous administration may result in the blood plasma concentration of psilocin in the patient being greater than or equal to about 5 ng/mL for a time of less than about 340 minutes, or less than about 320 minutes, or less than about 300 minutes, or less than about 280 minutes, or less than about 260 minutes, or less than about 240 minutes, or less than about 220 minutes, or less than about 200 minutes, or less than about 180 minutes, or less than about 160 minutes, or less than about 140 minutes, or less than about 120 minutes, or less than about 100 minutes, or less than about 80 minutes, or less than about 60 minutes, or less than about 40 minutes.

Administering the therapeutically effective amount of the psychedelic compound to the patient by subcutaneous injection may achieve a blood plasma concentration of psilocin in the patent of greater than or equal to about 5 ng/mL for a time of from about 40 to about 300 minutes, or from about 50 to about 280 minutes, for from about 60 to about 260 minutes, or from about 70 to about 240 minutes, or from about 80 to about 220 minutes. Typically, administering the therapeutically effective amount of the psychedelic compound to the patient by subcutaneous injection results in the blood plasma concentration of psilocin in the patient being greater than or equal to about 5 ng/mL for a time of from about 100 to about 200 minutes.

When the method of the invention achieves a blood plasma concentration of psilocin of greater than or equal to about 5 ng/mL in a time from the subcutaneous injection of less than about 20 minutes, it may take less than about 100 minutes for the blood plasma concentration of psilocin to return to about 5 ng/mL or less. When the method of the invention achieves a blood plasma concentration of psilocin of greater than or equal to about 5 ng/mL in a time from the subcutaneous injection of less than about 18 minutes, it may take less than about 120 minutes for the blood plasma concentration of psilocin to return to about 5 ng/mL or less. When the method of the invention achieves a blood plasma concentration of psilocin of greater than or equal to about 5 ng/mL in a time from the subcutaneous injection of less than about 16 minutes, it may take less than about 140 minutes for the blood plasma concentration of psilocin to return to about 5 ng/mL or less. When the method of the invention achieves a blood plasma concentration of psilocin of greater than or equal to about 5 ng/mL in a time from the subcutaneous injection of less than about 14 minutes, it may take less than about 160 minutes for the blood plasma concentration of psilocin to return to about 5 ng/mL or less. When the method of the invention achieves a blood plasma concentration of psilocin of greater than or equal to about 5 ng/mL in a time from the subcutaneous injection of less than about 12 minutes, it may take less than about 170 minutes for the blood plasma concentration of psilocin to return to about 5 ng/mL or less. When the method of the invention achieves a blood plasma concentration of psilocin of greater than or equal to about 5 ng/mL in a time from the subcutaneous injection of less than about 10 minutes, it may take less than about 180 minutes for the blood plasma concentration of psilocin to return to about 5 ng/mL or less. When the method of the invention achieves a blood plasma concentration of psilocin of greater than or equal to about 5 ng/mL in a time from the subcutaneous injection of less than about 8 minutes, it may take less than about 190 minutes for the blood plasma concentration of psilocin to return to about 5 ng/mL or less. When the method of the invention achieves a blood plasma concentration of psilocin of greater than or equal to about 5 ng/mL in a time from the subcutaneous injection of less than about 6 minutes, it may take less than about 200 minutes for the blood plasma concentration of psilocin to return to about 5 ng/mL or less. When the method of the invention achieves a blood plasma concentration of psilocin of greater than or equal to about 5 ng/mL in a time from the subcutaneous injection of less than about 4 minutes, it may take less than about 210 minutes for the blood plasma concentration of psilocin to return to about 5 ng/mL or less. When the method of the invention achieves a blood plasma concentration of psilocin of greater than or equal to about 5 ng/mL in a time from the subcutaneous injection of less than about 2 minutes, it may take less than about 220 minutes for the blood plasma concentration of psilocin to return to about 5 ng/mL or less.

An advantageous pharmacokinetic profile may also be obtained by administering a controlled release dosage form of the psychedelic compound to a patient by subcutaneous injection as discussed herein. Such controlled release dosage forms may comprise a pharmaceutical composition comprising the psychedelic compound, which pharmaceutical composition is a formulation selected from a suspension, emulsion, gel, liposome poorly soluble salt formulation, oily depot, viscous depot, protein binding system, lipidic system, polymer system, particulate system, or an in-situ gelling system.

For example, when a controlled release dosage form is used, the absorption half-life of the psychedelic compound in the controlled release dosage form may be from about 0.5 minutes to about 200 minutes, from about 1 minute to about 170 minutes, from about 2 minutes to about 140 minutes, from about 3 minutes to about 110 minutes, or from about 4 minutes to about 80 minutes.

As used herein, the term “absorption half-life of the psychedelic compound” refers to the time taken for 50% of a given dose of drug to be absorbed into the systemic circulation.

Administration of Two or More Doses of the Psychedelic Compound

An improved PK profile of psilocin can also be obtained by administering two or more doses of the psychedelic compound by subcutaneous injection, wherein the two or more doses are of different quantities and/or have different absorption half-lives of the psychedelic compound. If more than one injection is administered, “dose” refers to the mass of psychedelic compound given on each injection. If one injection is administered, “dose” refers to the mass of one fraction of the psychedelic compound given in the injection, wherein the absorption rate of that fraction is different to the absorption rate of another fraction of the psychedelic compound administered.

Thus, administering the therapeutically effective amount of the psychedelic compound to the patient by subcutaneous injection may comprise administering: a first dose of the psychedelic compound with a first absorption half-life of the psychedelic compound of (t_{firstdose})_{1/2}; and a second dose of the psychedelic compound with a second absorption half-life of the psychedelic compound of (t_{seconddose})_{1/2}. Typically, in this embodiment, (t_{seconddose})_{1/2}>(t_{firstdose})_{1/2}.

Typically, when two or more doses of the psychedelic compound are administered by subcutaneous injection, (t_{seconddose})_{1/2}>n (t_{firstdose})_{1/2}; and n=1, 2, 3, 4, 5, 6, 7, 8, 9, 10, 11, 12, 13, 14, 15, 16, 17, 18, 19, or 20.

The two or more doses of the psychedelic compound may be administered to the patient as a single subcutaneous injection. For example, the single subcutaneous injection may comprise two or more separate components each comprising a dose of the psychedelic compound. The single subcutaneous injection may comprise a formulation comprising a first component comprising a first dose of the psychedelic compound and a second component comprising a second dose of the psychedelic compound, wherein the first and second doses have different absorption half-lives.

For instance, the first component may be a rapid release component from which the first dose of the psychedelic compound is rapidly absorbed and the second component may be a delayed release component from which the second dose of the psychedelic compound is slowly absorbed. The formulation may for example be a suspension formulation comprising a first component which is a solution of the psychedelic compound and a second component which is solid particles comprising the psychedelic compound. The formulation may for example be a liposome formulation comprising a first component comprising the psychedelic compound encapsulated in a liposome and a second component comprising non-encapsulated psychedelic compound.

Alternatively, the two or more doses of the psychedelic compound may be administered to the patient as two or more separate subcutaneous injections, which may be administered at substantially the same time, or may be administered at different times. If the two or more subcutaneous injections are administered at different times, they may be administered from a single syringe or equivalent device.

The first dose of the psychedelic compound may be from about 0.1 mg to about 10 mg, from about 0.2 mg to about 9 mg, from about 0.3 mg to about 8 mg, from about 0.4 mg to about 7 mg, from about 0.5 mg to about 6 mg, from about 0.6 mg to about 5 mg, from about 0.7 mg to about 4 mg, from about 0.8 mg to about 3 mg, from about 0.9 mg to about 2 mg, or from about 1 mg to about 1.5 mg. In one embodiment, the psychedelic compound is psilocybin and the first dose is from about 1.0 mg to about 1.5 mg. In another embodiment, the psychedelic compound is psilocin and the first dose is from about 0.7 mg to about 1.1 mg.

The first dose of the psychedelic compound may be from about from about 0.1 mg to about 0.3 mg, from about 0.3 mg to about 0.5 mg, from about 0.5 mg to about 0.7 mg, from about 0.7 mg to about 0.9 mg, from about 0.9 mg to about 1.1 mg, from about 1.1 mg to about 1.3 mg, from about 1.3 mg to about 1.5 mg, from about 1.5 mg to about 1.7 mg, or from about 1.7 mg to about 1.9 mg.

The first dose is typically less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose.

The first dose of the psychedelic compound may be from about from about 0.1 mg to about 0.3 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 0.3 mg to about 0.5 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 0.5 mg to about 0.7 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 0.7 mg to about 0.9 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 0.9 mg to about 1.1 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 1.1 mg to about 1.3 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 1.3 mg to about 1.5 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 1.5 mg to about 1.7 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 1.7 mg to about 1.9 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose.

Typically, the psychedelic compound is psilocybin and the total dose administered may be from about 6 mg to about 11 mg, with from about 10% to about 35% of the total dose being administered as the first dose, or the total dose of psilocybin administered may be from about 7 mg to about 10 mg, with from about 20% to about 35% of the total dose being administered as the first dose. Alternatively, the psychedelic compound is psilocin and the total dose administered may be from about 4 mg to about 8 mg, with from about 10% to about 35% of the total dose being administered as the first dose, or the total dose of psilocin administered may be from about 5 mg to about 7 mg, with from about 20% to about 35% of the total dose being administered as the first dose.

(t_{firstdose})_{1/2} may from about 0.5 minutes to about 30 minutes, from about 0.5 minutes to about 15 minutes, from about 1 minute to about 8 minutes, from about 2 minutes to about 7 minutes, from about 3 minutes to about 6 minutes, or from about 4 minutes to about 5 minutes.

(t_{seconddose})_{1/2} may be from about 1 minute to about 250 minutes, about 5 minutes to about 200 minutes, from about 10 minutes to about 170 minutes, from about 15 minutes to about 140 minutes, or from about 20 minutes to about 110 minutes.

The method may further comprise administering a m^{th} dose of said psychedelic compound with a m^{th} absorption half-life of the psychedelic compound of (t_{mthdose})_{1/2}, wherein m is 3, 4, 5, 6, 7, 8, 9, or 10. For example, the method may comprise administering a third dose of said psychedelic compound with a third absorption half-life of the psychedelic compound of (t_{thirddose})_{1/2}. The m^{th} dose may be administered together with the first and second doses as a single subcutaneous injection.

The absorption of the first, second and/or subsequent doses into systemic circulation may follow first-order kinetics, or may vary according to zero order, quadratic, or any other kinetics.

Alternatively, the doses of the psychedelic compound may be administered to the patient as two or more separate subcutaneous injections.

The first, second or further dosage forms of the psychedelic compound may be formulated as a suspension, emulsion, gel, liposome poorly soluble salt formulation, oily depot, viscous depot, protein binding system, lipidic system, polymer system, particulate system, or an in-situ gelling system.

Compositions

As discussed herein, the invention may comprise administering a pharmaceutical composition comprising a therapeutically effective amount of the psychedelic compound to a patient. The pharmaceutical composition is typically a formulation suitable for subcutaneous injection. For example, the pharmaceutical composition may comprise the psychedelic compound and one or more pharmaceutically acceptable carriers, solvents, diluents, adjuvants, excipients, or vehicles. Typically, the pharmaceutical composition is a formulation comprising the psychedelic compound, at least one pharmaceutically acceptable diluent, and optionally further comprising at least one pharmaceutically acceptable buffer, solubiliser, polymer, and/or surfactant. The pharmaceutical composition may be a solution in which the psychedelic compound is dissolved. When the pharmaceutical composition is a solution, it is preferably an aqueous solution. The pharmaceutical composition may further comprise one or more of a buffer, a solubilizer, a pH modifier, a surfactant, an anti-oxidant, a chelating agent, a cyclodextrin, an oil, a polymer, or a phospholipid. Typical parenteral media may be used, for example water for injection, saline, dextrose etc.

The diluent is typically selected from water, saline, phosphate-buffered saline (PBS) and corn oil.

The pharmaceutical composition may be formulated as a solution, suspension, emulsion, gel, liposome poorly soluble salt formulation, oily depot, viscous depot, protein binding system, lipidic system, polymer system, particulate system, or an in-situ gelling system comprising the psychedelic compound. Typically, the pharmaceutical composition is formulated as a solution or as a suspension. In one embodiment, the formulation is a solution wherein the psychedelic compound is dissolved in a diluent. In one embodiment, the formulation is a suspension. The suspension comprises particles comprising the psychedelic compound suspended in a diluent.

The formulation may comprise a single component comprising the psychedelic compound. For example, if the formulation is a suspension, substantially all of the psychedelic compound may be present in the form of suspended solid particles of the psychedelic compound. Alternatively, the formulation may comprise two or more components each comprising the psychedelic compound. The formulation may for example be a suspension formulation comprising a first component which is a solution of the psychedelic compound and a second component which is solid particles comprising the psychedelic compound. The formulation may for example be a liposome formulation comprising a first component comprising the psychedelic compound encapsulated in a liposome and a second component comprising non-encapsulated psychedelic compound.

The formulation may comprise the psychedelic compound at a concentration of at least about 50 mg/g relative to the total weight of the formulation. Typically, the formulation comprises the psychedelic compound at a concentration of at least about 60 mg/g, at least about 70 mg/g, at least about 80 mg/g, at least about 90 mg/g, at least about 100 mg/g, at least about 125 mg/g, at least about 150 mg/g, at least about 175 mg/g, at least about 200 mg/g, or at least about 225 mg/g. Preferably, the formulation comprises the psychedelic compound at a concentration of at least about 70 mg/g, or at least about 80 mg/g, or at least about 90 mg/g. The formulation may comprise the psychedelic compound at a concentration of no more than about 400 mg/g, preferably no more than about 300 mg/g. For instance, the concentration of the psychedelic compound may be from 70 to 300 mg/g relative to the total weight of the formulation. The concentration of the psychedelic compound may be from 70 to 100 mg/g, from 100 to 130 mg/g, from 130 to 170 mg/g, from 170 to 220 mg/g or from 220 to 300 mg/g.

When the psychedelic compound is psilocybin or a pharmaceutically acceptable salt thereof, typically the formulation comprises the psychedelic compound at a concentration of at least about 60 mg/g, at least about 70 mg/g, at least about 80 mg/g or at least about 90 mg/g. Preferably, when the psychedelic compound is psilocybin or a pharmaceutically acceptable salt thereof, the formulation comprises psilocybin at a concentration of at least about 70 mg/g, more preferably at least about 80 mg/g. When the psychedelic compound is psilocybin or a pharmaceutically acceptable salt thereof, the formulation may comprise psilocybin at a concentration of about 90 mg/g, for instance from 80 to 100 mg/g.

When the psychedelic compound is psilocin or a pharmaceutically acceptable salt thereof, typically the formulation comprises the psychedelic compound at a concentration of at least about 150 mg/g, at least about 175 mg/g, at least about 200 mg/g, at least about 225 mg/g, or at least about 250 mg/g. Preferably, when the psychedelic compound is psilocin or a pharmaceutically acceptable salt thereof, the formulation comprises psilocin at a concentration of at least about 200 mg/g, more preferably at least about 225 mg/g. When the psychedelic compound is psilocin or a pharmaceutically acceptable salt thereof, the formulation may comprise psilocin at a concentration of about 250 mg/g, for instance from 230 to 270 mg/g.

Where the formulation comprises solid particles of the psychedelic compound (for example, where the formulation is a suspension), the particles of the psychedelic compound in the formulation may have particle size distribution with a D₅₀ of less than about 5 μm. Typically, the particles of the psychedelic compound have a D₅₀ of less than about 4 μm, less than about 3 μm, less than about 2 μm or less than about 1.5 μm. Preferably, the particles of the psychedelic compound have a D₅₀ of less than about 3 μm, less than about 2 μm or less than about 1.5 μm. More preferably, the particles of the psychedelic compound have a D₅₀ of less than about 1.5 μm. The D₅₀ is typically at least 0.8 μm. For instance, the particles of the psychedelic compound may have a D₅₀ of from 0.9 to 1.5 μm or from 1.05 to 1.25 μm. When the psychedelic compound is psilocybin or a pharmaceutically acceptable salt thereof, the D₅₀ is typically from 1.1 to 1.2 μm. When the psychedelic compound is psilocin or a pharmaceutically acceptable salt thereof, the D₅₀ is typically from 1.0 to 1.1 μm.

The particles of the psychedelic compound may have a D₁₀ of from 0.2 to 1.0 μm, for instance from 0.3 to 0.6 μm.

The particles of the psychedelic compound may have a D₉₀ of from 1.5 to 10 μm, for instance from 2.0 to 4.0 μm. When the psychedelic compound is psilocybin or a pharmaceutically acceptable salt thereof, the D₉₀ is typically from 2.5 to 3.5 μm. When the psychedelic compound is psilocin or a pharmaceutically acceptable salt thereof, the D₉₀ is typically from 2.0 to 2.5 μm.

When the psychedelic compound is psilocybin or a pharmaceutically acceptable salt thereof, the particles of the psychedelic compound (for instance as present in a suspension) may have a D₁₀ of from 0.2 to 0.7 μm, a D₅₀ of from 1.0 to 1.4 μm and a D₉₀ of from 2.8 to 3.5 μm. When the psychedelic compound is psilocin or a pharmaceutically acceptable salt thereof, the particles of the psychedelic compound (for instance as present in a suspension) may have a D₁₀ of from 0.2 to 0.7 μm, a D₅₀ of from 0.9 to 1.3 μm and a D₉₀ of from 1.9 to 2.8 μm.

As used herein, particle size distributions are typically as measured using laser diffraction. For instance, the D₁₀, D₅₀ and D₉₀ values are typically as measured by laser diffraction using a particle size analyser with a dry dispersion unit. The D₁₀, D₅₀ and D₉₀ values are typically volume averages (i.e. Dv0, Dv50 and Dv90 values). The particles of the psychedelic compound typically comprise at least 50% by weight of the psychedelic compound. For instance, the particles of the psychedelic compound typically comprise at least 80% by weight of the psychedelic compound. The particles of the psychedelic compound may comprise at least 95% by weight of the psychedelic compound. The particles of the psychedelic compound may alternatively comprise at least 50%, at least 80% or at least 95% by weight of a co-crystal of the psychedelic compound.

The formulation may have a pH of from pH 3 to pH 9. Typically, the formulation has a pH of from pH 4 to pH 8, for example a pH of from pH 4 to pH 6, or from pH 6 to pH 8. When the formulation is a solution and the psychedelic compound is psilocin or a pharmaceutically acceptable salt thereof, typically the pH is from pH 3 to pH 5, and is preferably about pH 4.

In one embodiment, the formulation is a suspension comprising particles of the psychedelic compound, polyvinylpyrrolidone (for example PVP k17), polyoxyethylene (80) sorbitan monooleate (Tween 80), and phosphate-buffered saline, wherein the psychedelic compound is psilocybin. The formulation may comprise polyvinylpyrrolidone at a concentration of from 0.1 to 1.0% w/v, polyoxyethylene (80) sorbitan monooleate at a concentration of from 0.01 to 0.50% w/v, and phosphate-buffered saline at a concentration of at least 98.5% w/v. Preferably, the formulation comprises polyvinylpyrrolidone at a concentration of from 0.4 to 0.6% w/v, polyoxyethylene (80) sorbitan monooleate a concentration of from 0.05 to 0.20% w/v, and phosphate-buffered saline at a concentration of at least 98.5% w/v. More preferably, the formulation comprises polyvinylpyrrolidone at a concentration of from 0.4 to 0.6% w/v, polyoxyethylene (80) sorbitan monooleate a concentration of from 0.05 to 0.20% w/v, and phosphate-buffered saline at a concentration of at least 98.5% w/v, wherein psilocybin is present at a concentration of from 80 to 100 mg/g.

In one embodiment, the formulation is a suspension comprising particles of the psychedelic compound, 2-hydroxypropyl-β-cyclodextrin and phosphate-buffered saline, wherein the psychedelic compound is psilocybin. The formulation may comprise 2-hydroxypropyl-β-cyclodextrin at a concentration of from 5 to 15% w/v, and phosphate-buffered saline at a concentration of from 85 to 95% w/v. Preferably, the formulation comprises 2-hydroxypropyl-β-cyclodextrin at a concentration of from 8 to 12% w/v, and phosphate-buffered saline at a concentration of from 88 to 92% w/v. More preferably, the formulation comprises 2-hydroxypropyl-β-cyclodextrin at a concentration of from 8 to 12% w/v, and phosphate-buffered saline at a concentration of from 88 to 92% w/v, wherein psilocybin is present at a concentration of from 80 to 100 mg/g.

In one embodiment, the formulation is a suspension comprising particles of the psychedelic compound, polyethylene-polypropylene glycol (Poloxamer 188) and phosphate-buffered saline, wherein the psychedelic compound is psilocybin. The formulation may comprise polyethylene-polypropylene glycol at a concentration of from 1 to 5% w/v, and phosphate-buffered saline at a concentration of from 95 to 99% w/v. Preferably, the formulation comprises polyethylene-polypropylene glycol at a concentration of from 1 to 3% w/v, and phosphate-buffered saline at a concentration of from 97 to 99% w/v. More preferably, the formulation comprises polyethylene-polypropylene glycol at a concentration of from 1 to 3% w/v, and phosphate-buffered saline at a concentration of from 97 to 99% w/v, wherein psilocybin is present at a concentration of from 80 to 100 mg/g.

In one embodiment, the formulation is a solution comprising the psychedelic compound, 2-hydroxypropyl-β-cyclodextrin and water, wherein the psychedelic compound is psilocin. The formulation may comprise 2-hydroxypropyl-β-cyclodextrin at a concentration of from 5 to 15% w/v, and water at a concentration of from 85 to 95% w/v. Preferably, the formulation comprises 2-hydroxypropyl-β-cyclodextrin at a concentration of from 8 to 12% w/v, and water at a concentration of from 88 to 92% w/v. More preferably, the formulation comprises 2-hydroxypropyl-β-cyclodextrin at a concentration of from 8 to 12% w/v, and water at a concentration of from 88 to 92% w/v, wherein psilocin is present at a concentration of from 230 to 270 mg/g.

In one embodiment, the formulation is a solution comprising the psychedelic compound and water, wherein the psychedelic compound is psilocin. The formulation may consist of water and psilocin. The formulation may therefore comprise 100% w/v water. Preferably, the formulation comprises 100% w/v water, wherein psilocin is present at a concentration of from 230 to 270 mg/g.

In one embodiment, the formulation is a suspension comprising particles of the psychedelic compound, corn oil, N-methylpyrrolidone and polyoxyl-35-castor oil (Kolliphor ELP), wherein the psychedelic compound is psilocin. The formulation may comprise corn oil at a concentration of from 70 to 90% w/w, N-methylpyrrolidone at a concentration of from 5 to 15% w/w, and polyoxyl-35-castor oil at a concentration of from 5 to 15% w/w. Preferably, the formulation comprises corn oil at a concentration of from 76 to 84% w/w, N-methylpyrrolidone at a concentration of from 8 to 12% w/w, and polyoxyl-35-castor oil at a concentration of from 8 to 12% w/w. More preferably, the formulation comprises corn oil at a concentration of from 76 to 84% w/w, N-methylpyrrolidone at a concentration of from 8 to 12% w/w, and polyoxyl-35-castor oil at a concentration of from 8 to 12% w/w, wherein psilocin is present at a concentration of from 230 to 270 mg/g.

Concentrations of an excipient as used herein refer to the amount of that excipient relative to the total amount of excipient (i.e. excluding the psychedelic compound).

The formulations comprising psilocybin as described herein are typically stable for 7 days at 25° C. The formulations comprising psilocin as described herein are typically stable for 7 days at 2-8° C. As used herein, “stable” refers to stability with respect to chemical degradation of the psychedelic compound.

Typically, the method of the invention comprises administering a pharmaceutical composition comprising a therapeutically effective amount of the psychedelic compound to a patient by subcutaneous injection, which pharmaceutical composition is a formulation as described herein.

When the method of the invention comprises administering a pharmaceutical composition to a patient by subcutaneous injection, the method may further comprise producing the formulation suitable for subcutaneous injection by (a) defrosting a frozen solution, (b) reconstituting a lyophilised product, (c) solubilising a powder, or (d) diluting a concentrate.

Suitable carriers, solvents, diluents, adjuvants, excipients, vehicles, buffers, solubilizers, pH modifiers, surfactants, anti-oxidants, chelating agents, cyclodextrins, oils, polymers, and phospholipids are generally well known to the skilled person, and can be found in standard pharmaceutical texts.

Methods of Treatment

The method of the present invention is suitable for treating any disease or condition that psychedelic compounds, such as psilocybin or psilocin, may be used to treat.

In one embodiment, the invention provides a method of treating or preventing a disease or condition selected from psychological, neurological and central nervous system disorders.

The disease or condition may be selected from: disruptive mood dysregulation disorder, depression, major depressive disorder (MDD), treatment-resistant depression, persistent depressive disorder (dysthymia), demoralization, hopelessness, premenstrual dysphoric disorder, substance/medication-induced depressive disorder, post-partum depression, depressive disorder due to another medical condition, separation anxiety disorder, selective mutism, specific phobia, social anxiety disorder (social phobia), panic disorder, panic attack, agoraphobia, generalized anxiety disorder, anxiety, death anxiety, substance-medication-induced anxiety disorder, anxiety disorder due to another medical condition, somatic symptom disorder, illness anxiety disorder (hypochondriac), conversion disorder (functional neurological symptom disorder), factitious disorder, post-traumatic stress disorder (PTSD), adjustment disorders, acute distress disorder, obsessive-compulsive disorder, body dysmorphic disorder, hoarding disorder, trichotillomania (hair-pulling) disorder, excoriation (skin-picking) disorder, substance/medication-induced obsessive-compulsive and related disorder, obsessive-compulsive and related disorder due to another medical condition, substance-related disorders, alcohol-related disorders, cannabis-related disorders, hallucinogen-related disorders, inhalant-related disorders, cocaine-related disorders, opioid-related disorders, sedative-, hypnotic-, or anxiolytic-related disorders, stimulant-related disorders, tobacco-related disorders, non-substance-related disorders (gambling or gaming disorder), migraines, cluster headaches (including chronic cluster headaches), cyclical vomiting, tension-type headache, dysphasia, pica, anorexia nervosa, bulimia nervosa, binge-eating disorder, oppositional defiant disorder, intermittent explosive disorder, conduct disorder, antisocial personality disorder, psychopathy, pyromania, kleptomania, autism spectrum disorder, antisocial personality disorder, attention-deficit/hyperactivity disorder, schizotypal (personality) disorder, delusional disorder, schizophrenia, schizoaffective disorder, insomnia disorder, hypersomnolence disorder, narcolepsy, primary central sleep apnea, bipolar I disorder, bipolar II disorder, cyclothymic disorder, pain, phantom pain, chronic pain, myelopathy, traumatic brain injury, intellectual disabilities, mania, neurodegeneration, paraphilic disorders (e.g., paedophilic disorder), suicidal behavior disorder, suicidal ideation, desire for hastened death, non-suicidal self-injury, persistent complex bereavement disorder, epilepsy, locked-in syndrome and restless leg syndrome.

In one embodiment, the method is a method of treating or preventing a disease or condition selected from depression, anxiety, death anxiety, demoralization, adjustment disorders, hopelessness, suicidal ideation and desire for hastened death.

In one embodiment, the method is a method of treating or preventing cocaine-related disorders, opioid-related disorders, or stimulant-related disorders.

The method may be a method of treating or preventing depression in a patient. The method may be a method of treating or preventing anxiety in a patient. As used herein, treating or preventing depression and/or anxiety includes reducing the symptoms of depression and/or anxiety or achieving remission of depression and/or anxiety. In one embodiment, treating or preventing depression and/or anxiety comprises reducing the symptoms of depression and/or anxiety. The patient may report a reduction of symptoms of depression and/or anxiety.

In one embodiment, the patient has been identified as being in need of treatment to alleviate depression and/or anxiety. In one embodiment, the patient has indicated that he or she is suffering from depression and/or anxiety.

The symptoms of depression and/or anxiety may be measured using the Hospital Anxiety and Depression Scale (HADS; Zigmond and Snaith (1983), “The hospital anxiety and depression Scale”, Acta Psychiatrica Scand, 67: 361-370). In this test, lower numbers indicate lower levels of depression and/or anxiety. Subscale scores can be calculated for depression (HADS-D) and anxiety (HADS-A). A subscale score equal to or above 8 and a full scale score over 12 indicates the possible presence of a clinical disorder.

Accordingly, in the method of the invention a total Hospital Anxiety and Depression Scale score of the patient may be reduced after administration of the therapeutically effective amount of the psychedelic compound. In one embodiment, the total HADS score of the patient is reduced to below about 12 after administration of the therapeutically effective amount of the psychedelic compound.

The severity of depression may also be measured using the Beck Depression Inventory-II (BDI-II; Beck et al (1988), “Psychometric properties of the Beck Depression Inventory: Twenty-five years of evaluation”, Clin Psych Rev, 8: 77-100). Scores above 12 indicate possible clinical depression.

Accordingly, in the method of the invention a Beck Depression Inventory-II score of the patient may be reduced after administration of the therapeutically effective amount of the psychedelic compound. In one embodiment, the Beck Depression Inventory-II score of the patient is reduced to below about 12 after administration of the therapeutically effective amount of the psychedelic compound.

The method may be a method of treating or preventing death anxiety. The method may be a method of treating or preventing demoralization (i.e. loss of meaning in life). The method may be a method of treating or preventing hopelessness. Death anxiety, demoralization and hopelessness are aspects of existential distress. Thus, the method may also be a method of treating or preventing existential distress in a patient, wherein treating or preventing existential distress includes reducing levels of at least one of death anxiety, hopelessness and demoralization.

In the method of treating or preventing death anxiety in a patient, death anxiety is reduced relative to the death anxiety of the patient before the administration of the therapeutically effective amount of the psychedelic compound. Death anxiety is typically measured according to the Death Anxiety Scale (Templer (1970), “The construction and validation of a death anxiety scale”, J Gen Psychol, 82: 165-177). Scores below 8 are considered normative levels of death anxiety. Accordingly, in the method of the invention a death anxiety score of the patient may be reduced to less than 8 after administration of the therapeutically effective amount of the psychedelic compound.

In the method of treating or preventing demoralization in a patient, demoralization is reduced relative to the demoralization of the patient before the administration of the therapeutically effective amount of the psychedelic compound. Demoralization is typically measured according to the Demoralization Scale (Kissane et al. (2004), “The demoralization scale: A report of its development and preliminary validation”, J Palliat Care, 20: 269-276). Scores above 30 are considered indicative of clinical levels of demoralization. Accordingly, in the method of the invention a demoralization score of the patient may be reduced to less than 30 after administration of the therapeutically effective amount of the psychedelic compound.

In the method of treating or preventing hopelessness in a patient, hopelessness is reduced relative to the hopelessness of the patient before the administration of the therapeutically effective amount of the psychedelic compound. Hopelessness is typically measured according to the Hopelessness Assessment in Illness instrument (Rosenfeld et al. (2011), “Assessing hopelessness in terminally ill cancer patients: Development of the Hopelessness Assessment in Illness Questionnaire”, Psychol Assess, 23: 325-336), on a scale of 0-16. Higher scores indicate higher levels of hopelessness. Accordingly, in the method of the invention a Hopelessness Assessment in Illness score of the patient may be reduced to less than 8 after administration of the therapeutically effective amount of the psychedelic compound.

The method may be a method of treating or preventing suicidal ideation in a patient. As used herein, treating or preventing suicidal ideation includes reducing or preventing suicidal thinking, suicidal planning and/or suicide attempts. The patient may report a reduction in suicidal thinking and/or suicidal planning. The patient may make less frequent suicide attempts.

In one embodiment, the patient has been identified as being in need of treatment to prevent or reduce suicidal ideation. Accordingly, the method of the invention may include a step of assessing the level of suicidal ideation in the patient prior to administering the therapeutically effective amount of the psychedelic compound to said patient. In one embodiment, the patient has indicated that he or she is suffering from suicidal ideation.

Suicidal ideation may be measured using a composite test comprising elements from the Beck Depression Inventory-II (BDI-II; Beck et al (1988), “Psychometric properties of the Beck Depression Inventory: Twenty-five years of evaluation”, Clin Psych Rev, 8: 77-100) and the Brief Symptom Inventory (BSI; Derogatis 1993). In the BDI, Item #9 queries suicidal ideation with the following options: 0=I don't have any thoughts of killing myself; 1=I have thoughts of killing myself, but I would not carry them out; 2=I would like to kill myself; 3=I would kill myself if I had the chance. In the BSI, item #9 (“Thoughts of ending your life”) also correlates to suicidal ideation, and is measured on a Likert scale: 0=Not at all; 1=Little; 2=Moderately; 3=Quite a bit; 4=Extremely. The aggregate composite suicidal ideation score is calculated by adding the scores from BDI-II item #9 to BSI Item #9. The composite score may be calculated by computing Z-scores for each item and summing them, and then the composite Z-scores may be transformed into standardized T-scores with a range of 0 to 100 (Song et al., 2013). Higher scores indicate higher SI.

Accordingly, in the method of the invention a composite suicidal ideation score of the patient may be reduced after administration of the therapeutically effective amount of the psychedelic compound. Typically, a composite suicidal ideation score of the patient is reduced by at least 20%, at least 30%, at least 40%, at least 50% or at least 75% after administration of the therapeutically acceptable amount of the psychedelic compound. In one embodiment, a composite suicidal ideation score of the patient after administration of the therapeutically effective amount of the psychedelic compound is less than 50, less than 45 or less than 40.

The method may be a method of treating or preventing desire for hastened death in a patient. As used herein, treating or preventing desire for hastened death includes preventing or reducing the desire for a more rapid death than would naturally occur. The patient may report a reduction in desire for a more rapid death than would naturally occur.

In one embodiment, the patient has been identified as being in need of treatment to prevent or reduce desire for hastened death. Accordingly, the method of the invention may include a step of assessing the level of desire for hastened death in the patient prior to administering the therapeutically effective amount of the psychedelic compound to said patient. In one embodiment, the patient has indicated that he or she is suffering from desire for hastened death.

Desire for hastened death may be measured using the schedule of attitudes towards hastened death (SAHD) (Rosenfeld 2000). The SAHD is a 20-item true/false measure of desire for hastened death, which has been validated in patients with cancer. Alternatively, DHD can be measured using the loss of meaning factor from the Demoralization Scale (Kissane et al. (2004)). In particular, a composite desire for hastened death score can be created from the following five items from the loss of meaning factor, as measured on a Likert scale from zero to four: “Life is no longer worth living”, “I would rather not be alive”, “My life seems to be pointless”, “My role in life has been lost”, and “There is no purpose to the activities in my life”.

Accordingly, in the method of the invention a composite desire for hastened death score of the patient may be reduced after administration of the therapeutically effective amount of the psychedelic compound. Typically, a composite desire for hastened death score of the patient is reduced by at least 20%, at least 40%, at least 60% or at least 80% after administration of the therapeutically effective amount of the psychedelic compound.

The patient to be treated may be suffering from a life-threatening disease. The life-threatening disease may be any chronic disease which has the potential to reduce the normal life expectancy of a patient suffering from the disease. The life-threatening disease may be selected from cancer, heart disease, chronic obstructive pulmonary disease (COPD), diabetes mellitus, Alzheimer's, dementia, motor neurone disease, amyotrophic lateral sclerosis (ALS), Parkinson's disease, epilepsy, multiple sclerosis, and myalgic encephalopathy (ME). In one embodiment, the life-threatening disease is cancer.

Also provided by the invention is psychedelic compound for use in a method of treating or preventing a disease or condition in a patient, wherein the psychedelic compound is psilocybin or psilocin, or a pharmaceutically acceptable salt thereof; and the method comprises administering a therapeutically effective amount of the psychedelic compound to the patient by subcutaneous injection.

In one embodiment, the invention provides a psychedelic compound as described herein, for use in the treatment of a disease or condition selected from a psychological, neurological and central nervous system disorder, and preferably selected from depression, anxiety, death anxiety, demoralization, adjustment disorders, hopelessness, suicidal ideation and desire for hastened death. In one embodiment, the disease or condition is selected from cocaine-related disorders, opioid-related disorders and stimulant-related disorders.

The invention further provides use of a psychedelic compound in the manufacture of a medicament for use in a method of treating or preventing of a disease or condition in a patient, wherein the psychedelic compound is psilocybin or psilocin, or a pharmaceutically acceptable salt thereof; and the method comprises administering a therapeutically effective amount of a psychedelic compound to the patient by subcutaneous injection. The disease or condition may be selected, for example, from a psychological, neurological and central nervous system disorder, such as depression, anxiety, death anxiety, demoralization, adjustment disorders, hopelessness, suicidal ideation and desire for hastened death. In one embodiment, the disease or condition is selected from cocaine-related disorders, opioid-related disorders and stimulant-related disorders. The treatment or prevention of the disease or condition may be as described herein.

Kits

The invention also provides a kit comprising one or more formulations suitable for administration by subcutaneous injection, which one or more formulations comprise a psychedelic compound which is psilocybin or psilocin, or a pharmaceutically acceptable salt thereof; and instructions for use of the one or more formulations in a method as defined herein.

The one or more formulations may be selected, for example, from a solution, suspension, emulsion, gel, liposome poorly soluble salt oily depos, viscous depot, protein binding systems, lipidic system, polymer system, particulate system, or an in-situ gelling system. The one or more formulations may be any of the formulations defined herein.

As used herein, the term “absorption half-life of the psychedelic compound” refers to the time taken for 50% of a given dose of drug to be absorbed into the systemic circulation.

The invention is described in more detail by the following Examples. Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

The texts of references cited in this disclosure are herein incorporated by reference in their entireties.

### EXAMPLES

### Example 1

All analysis was performed using the software R and RStudio, with models being defined using analytical and ODE solutions.

Derivation of system PK parameters for psilocybin/psilocin Systemic PK parameters were derived for published human bolus psilocybin IV data.

Human IV PK data for psilocin after psilocybin was administered was taken from Hasler et al. “Determination of psilocin and 4-hydroxyindole-3-acetic acid in plasma by HPLC-ECD and pharmacokinetic profiles of oral and intravenous psilocybin in man”. Pharm Acta Helv. 1997 June; 72(3):175-84. Model selection was performed by assessing visual fit to data and the respective model AIC's of a one compartment and two compartment IV PK model. These systemic human PK parameters were then used for human intramuscular injection simulations by simulating the PK profiles over a range of doses and absorption rates.

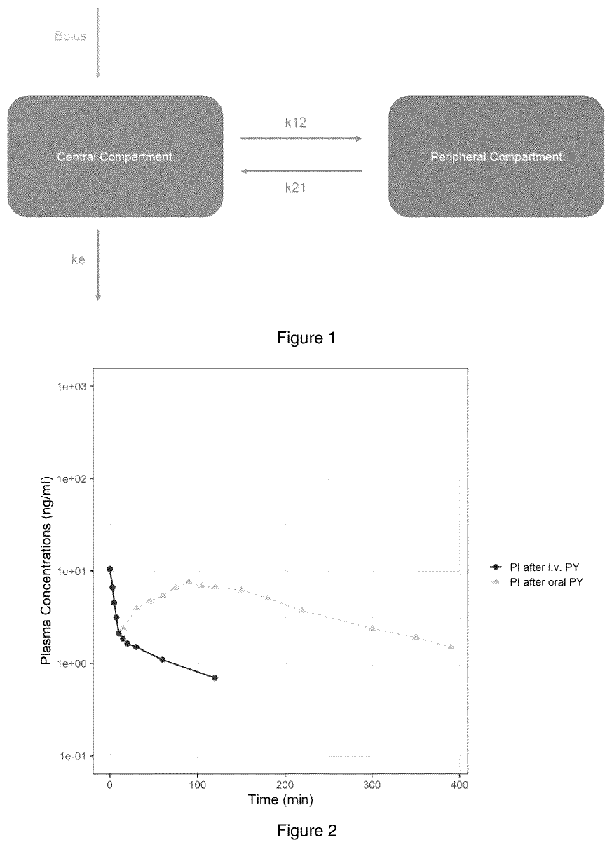

A two-compartment IV analytical model best described the data as seen in FIG. 1 and equations (1)-(6), Cl is the clearance (units mL/min), V is the volume of distribution of the central compartment (units mL), with k12, k21 (units 1/min) representing the distribution rates to and from the peripheral compartment.

$$ke = Cl / V , \qquad ( 1 )$$

$$beta = 0.5 * ( k 12 + k 21 + k e - s q r t ( {( k 12 + k 21 + k e )}^{2} - 4 * k 21 * k e ) ) , \qquad ( 2 )$$

$$alpha = k 21 * \frac{ke}{beta} , \qquad ( 3 )$$

$$A = \frac{alpha - k 21}{V * ( a l p h a - b e t a )} , \qquad ( 4 )$$

$$B = \frac{beta - k 21}{V * ( beta - alpha )} , \qquad ( 5 )$$

$$Conc = dose * ( A * exp ( - alpha * t 1 ) + B * exp ( - beta * t 1 ) ) \qquad ( 6 )$$

FIG. 2 shows visualized plasma concentration data (Hasler et al. “Determination of psilocin and 4-hydroxyindole-3-acetic acid in plasma by HPLC-ECD and pharmacokinetic profiles of oral and intravenous psilocybin in man”. Pharm Acta Helv. 1997 June; 72(3):175-84). It is assumed that the generation of psilocin is instantaneous and complete once psilocybin is absorbed. FIG. 2 shows measured exposure of psilocin after a 1 mg psilocybin IV administration and psilocin levels after a 15 mg oral dose of psilocybin.

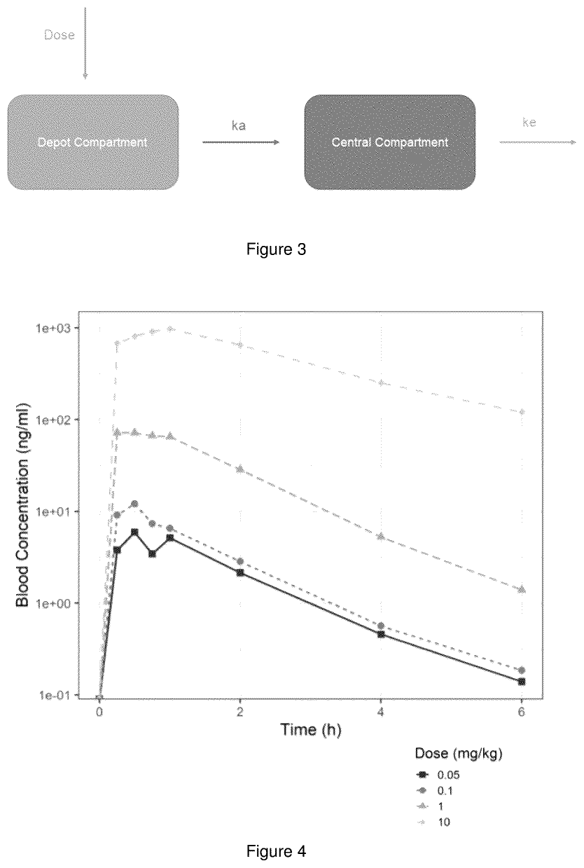

An IV two compartment model best described the IV data from FIG. 2. A two-compartment model gave a good description of the raw data capturing the Cₘₐₓ and terminal phase well, FIG. 3. The systemic PK parameters can be seen in Table 1 and there are low errors in the parameter estimates.

TABLE 1
Fitted values of two compartment IV model

| Parameter | Parameter estimate (95% Cl) |
|---|---|
| V (ml) | 88600 (77300, 99900) |
| Cl (ml/min) | 3810 (3340, 4290) |
| k12 (/min) | 0.176 (0.144, 0.208) |
| k21 (/min) | 0.0489 (0.0388, 0.0591) |

Rat Subcutaneous Absorption Data for Psilocybin/Psilocin

Rat SC PK data was digitised for four dose levels (Higgins, G. A. et al., 2021. “Low Doses of Psilocybin and Ketamine Enhance Motivation and Attention in Poor Performing Rats: Evidence for an Antidepressant Property”. Frontiers in Pharmacology, Volume 12, p. 299). The PK was fitted using a one compartment SC model where volume was fixed to the same as human in L/kg. The model was fit to the lower 3 doses levels as the higher dose was determined to be out of the range of investigation.

Simulation Design and Results

A one compartment model was used with linear absorption and clearance shown schematically in FIG. 3. Mathematically, this model is a series of two linked ordinary differential equations (ODE's) given in equations (7)-(8).

$$\frac{d}{d t} depot = - ka * depot , \qquad ( 7 )$$

$$\frac{d}{dt} centre = ka * depot - \frac{Cl}{Vc} * centre \qquad ( 8 )$$

Constant ka is the absorption rate constant (units 1/h), Cl is the clearance (units mL/h), Vc is the volume of distribution of the central compartment (units mL).

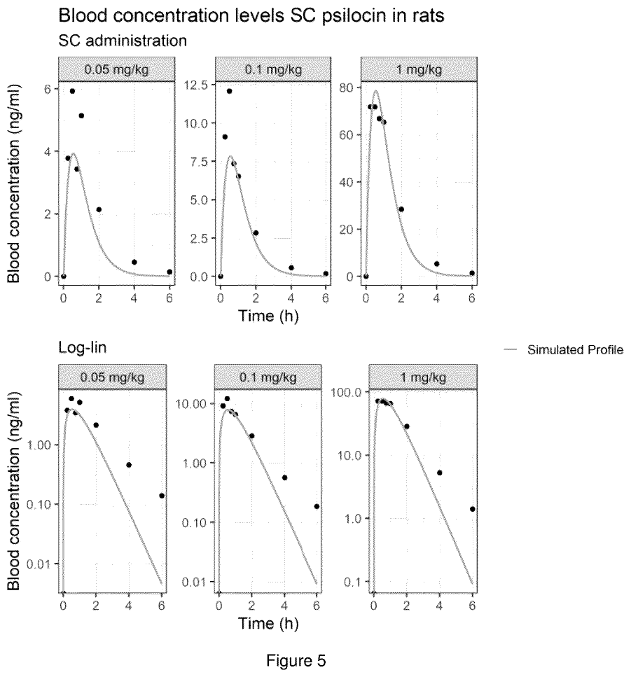

FIG. 4 shows psilocin blood concentration following subcutaneous administration of psilocybin to rats at 4 dose levels: 0.05 mg/kg, 0.1 mg/kg, 1 mg/kg and 10 mg/kg (Higgins, G. A. et al., 2021. “Low Doses of Psilocybin and Ketamine Enhance Motivation and Attention in Poor Performing Rats: Evidence for an Antidepressant Property”. Frontiers in Pharmacology, Volume 12, p. 299). Although there are four dose levels the PK model was only fit to the lower three doses, this is due to the dose normalised AUC of the 10 mg/kg dose being considerably greater than the other doses (not shown), the lower doses are also, in terms of mg/kg, closer to the doses the human simulations will be run on. It is assumed that the generation of psilocin is instantaneous once psilocybin is absorbed. Due to a lack of rat IV PK data and human and rat plasma protein binding data, total rat volume of distribution (V) on ml/kg basis was fixed to the same as human total volume of distribution. A one compartment SC model gives a satisfactory fit to the absorption phase, seen in FIG. 5, this gives good confidence of the fitted ka value seen in Table 2. Bioavailability was again assumed to be 1.

TABLE 2
Fitted one compartment SC parameters

| Parameter | Estimate (95% CI) |
|---|---|
| ka (/h) | 2.32 (2.03, 2.61) |
| Cl (mL/h) | 2490 (2260, 2710) |
| V (mL) | 1746 (Fixed) |

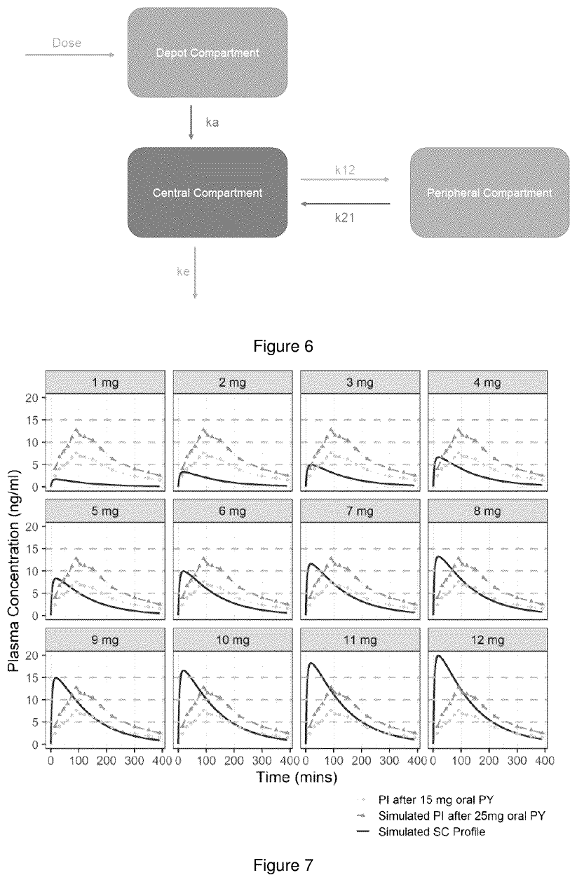

The model that was used is shown schematically in FIG. 6, mathematically the analytical solution is shown in equations (9)-(14).

$$ke = \frac{Cl}{V} , \qquad ( 9 )$$

$$beta = 0.5 * ( k 12 + k 21 + k e - s q r t ( {( k 12 + k 21 + k e )}^{2} - 4 * k 21 * k e ) ) , \qquad ( 10 )$$

$$alpha = \frac{k 21 * ke}{beta} , \qquad ( 11 )$$

$$A = ( ka / V ) * ( \frac{k 21 - alpha}{( beta - alpha ) * ( ka - alpha )} ) , \qquad ( 12 )$$

$$B = ( ka / V ) * ( \frac{k 21 - beta}{( alpha - beta ) * ( ka - beta )} ) , \qquad ( 13 )$$

$$Conc = dose * ( ( A * exp ( - alpha * t ) ) + ( B * exp ( - beta * t ) ) - ( ( A + B ) * exp ( - ka * t ) ) ) \qquad ( 14 )$$

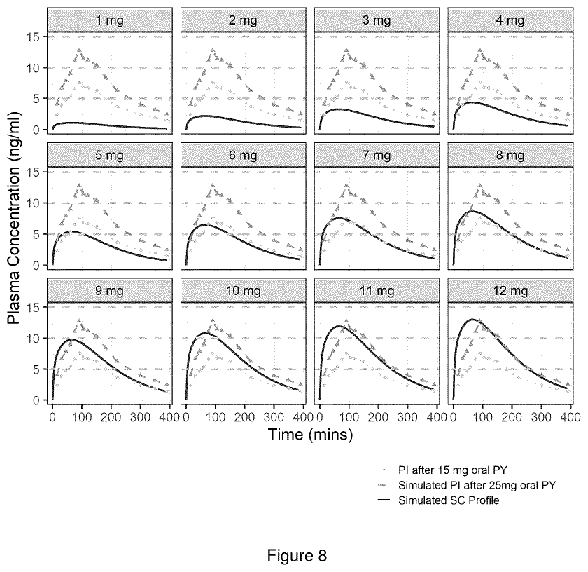

Using the absorption rate from modelling the rat SC data and the human systemic psilocin PK parameters, human SC simulations were run over 1-12 mg doses and the simulated PK profiles can be seen in FIG. 7. Table 3 shows the time to 5 ng/ml and the time above 5 ng/ml. The absorption half-life is 17.9 minutes. Increasing the absorption half-life by 2.5× to 44.8 minutes meant that a dose of 7-9 mg gave the target PK profile. These profiles can be seen in FIG. 8, with the summary statistics in Table 4.

TABLE 3
Table showing time to 5 ng/ml and time above 5 ng/ml
after a SC administration over a range of doses

| | | Time | |
|---|---|---|---|
| | Time to 5 | above 5 | |
| Dose | ng/ml | ng/ml | Exceeds 10 |
| (mg) | (min) | (min) | ng/ml |
| 4 | 5.2 | 66.9 | N |
| 5 | 3.5 | 98.5 | N |
| 6 | 2.7 | 122.7 | N |
| 7 | 2.2 | 142.6 | N |
| 8 | 1.8 | 159.6 | N |
| 9 | 1.6 | 174.5 | N |
| 10 | 1.4 | 187.7 | Y |
| 11 | 1.3 | 199.6 | Y |
| 12 | 1.1 | 210.6 | Y |

TABLE 4
Table showing time to 5 ng/ml and time
above 5 ng/ml after a SC administration
with 2.5x slower absorption rate over a range of doses

| | | Time | |
|---|---|---|---|
| | Time to 5 | above 5 | |
| Dose | ng/ml | ng/ml | Exceeds 10 |
| (mg) | (min) | (min) | ng/ml |
| 5 | 34 | 71.6 | N |
| 6 | 16 | 131.2 | N |
| 7 | 9.8 | 165.3 | N |
| 8 | 7.1 | 189.9 | N |
| 9 | 5.6 | 209.8 | N |
| 10 | 4.7 | 226.5 | Y |
| 11 | 4 | 241.1 | Y |
| 12 1 | 3.5 | 254.1 | Y |

### Example 2

The following Example describes the preparation of formulations suitable for administration by subcutaneous injection.

Materials

The materials used in the preparation of the formulations are as follows.

Material
Psilocybin
Psilocin
Methanol
Ethanol
Dimethyl sulfoxide (DMSO)
Dichloromethane (DCM)
N-methylpyrrolidone (NMP)
2H,3H-Decafluoropentane 98%
Orthophosphoric acid
Trifluoroacetic acid
Propylene glycol (PG)
Super refined Polysorbate 80-LQ-(MH) (Tween 80,
polyoxyethylene (80) sorbitan monooleate)
Super refined corn oil-LQ-(MH)
Super refined polyethylene glycol (PEG) 400-LQ-
(MH)
Kollidon ® 17 PF (polyvinylpyrrolidone, PVP k17)
Transcutol HP (2-(2-Ethoxyethoxy)ethanol)
Labrafac lipophile WL 1349 (mixture of triglycerides
of caprylic (C₈) and capric (C₁₀) acid)
Glycerine
Labrafil M 1944 CS (oleoyl macrogol-6 glycerides)
Kolliphor ® EL (polyoxyl-35-castor oil)
Labrafac PG (Propylene glycol dicaprylocaprate)
Kolliphor ® ELP (polyoxyl-35-castor oil)
Super refined peanut oil-LQ-(MH)
Kolliphor ® HS 15 (Polyethylene glycol 660 12-
hydoxystearate)
Super refined safflower USP-LQ-(MH)
Capryol 90 (Propylene glycol monocaprylate)
Sulfobutylether-β-Cyclodextrin (SBEβCD)
Cavitron ™W7 HP5 Pharma (2-Hydroxypropyl-β-
cyclodextrin, HPβCD)
Vitamin E TPGS
PEG 1500
Poloxamer 188 (Polyethylene-polypropylene glycol)
Hydrochloric acid 5M
Glacial acetic acid

Micronisation of Psychedelic Compound

In order to provide a small and homogeneous particle size of psychedelic compound in the final suspensions, psilocybin and psilocin were micronised before use. Micronisation was performed by wet bead milling using decafluoropentane as the antisolvent. In a glass vial, 200 mg psychedelic compound and 2 g of 1 mm silica beads were weighed. 3 mL of the antisolvent was added for psilocybin and 2 mL for psilocin. The samples were vortexed for approximately 15 seconds then milled at 530 rpm for 60 minutes.

The micronised sample was then separated from the silica beads and transferred to a petri dish and left at room temperature to allow for evaporation of the solvent overnight (approximately 18 hours). The dried sample was then collected and characterised by PSD and XRPD.

The PSD analysis results are shown in Table 5. XRPD analyses showed that both psilocybin and psilocin had no significant chemical degradation upon micronisation.

TABLE 5
PSD analysis results of feed and micronized psilocybin/psilocin.

| Sample | PSD parameters | D₁₀ | D₅₀ | D₉₀ |
|---|---|---|---|---|
| Feed | R3 lens, primary | 1.16 μm | 5.46 μm | 16.32 μm |
| psilocybin | pressure | | | |
| | of 4 bar | | | |
| Micronised | R1 lens, primary | 0.45 μm | 1.20 μm | 3.13 μm |
| psilocybin | pressure | | | |
| | of 4 bar | | | |
| Feed | R5 lens, primary | 20.36 μm | 106.05 μm | 232.08 μm |
| psilocin | pressure | | | |
| | of 4 bar | | | |
| Micronised | R1 lens, primary | 0.44 μm | 1.07 μm | 2.27 μm |
| psilocin | pressure | | | |
| | of 4 bar | | | |

Preparative Work

The solubility of non-micronised psilocybin and psilocin was first assessed in the following 20 excipients:

| DMSO | NMP |
|---|---|
| Ethanol | Propylene glycol |
| Tween 80 | PEG 400 |
| Transcutol HP | Glycerine |
| Kolliphor EL | Kolliphor HS 15 (10% aq. solution) |
| Capryol 90 | Labrafac lipophile WL 1349 |
| Labrafil M 1944 CS | Labrafac PG |
| Peanut oil | Safflower oil |
| Poloxamer 188 (10% aq. solution) | SBEβCD (10% aq. solution) |
| HPβCD (10% aq. solution) | Acetic acid (1% aq. solution) |
| PEG 1500 (10% aq. solution) | Vitamin E TPGS (10% aq. solution) |

Due to the poor solubility of psilocybin observed in all the excipients tested, suspension development was chosen as the most appropriate approach for psilocybin. Suspension development was investigated with non-micronised psilocybin in different aqueous vehicles (PVP k17/Tween 80, Kolliphor HS 15, Kolliphor ELP, HPβCD, and Poloxamer 188) and one non-aqueous vehicle (corn oil) at a starting concentration of 100 mg/g and visually assessed for flowability at higher concentrations up to 150 mg/g. A suspension formulation of micronized psilocybin in HPβCD was also prepared. Lead formulations were selected based on viscosity and initial stability measurements. It was decided to progress with the micronised psilocybin to ensure small and homogenous particle size in the final formulations. As the suspensions in water were hypotonic, it was decided to prepare the final formulations in PBS rather than water.

Psilocin was shown to have good solubility in DMSO and NMP. Suspension development and solution development by a co-solvency approach were therefore investigated. Solution development was investigated with psilocin at a concentration of 30 mg/g in different aqueous vehicles (PEG/Transcutol HP/HPβCD, DMSO/PEG 400/Kolliphor ELP) and one non-aqueous vehicle (DMSO/PEG 400/Kolliphor ELP, peanut oil). Initial trials illustrated that psilocin had higher solubility at acidic pH, and so based on the initial results the feasibility to develop solutions at higher concentrations of 200-250 mg/g psilocin, using low pH (pH 4), was assessed in the aqueous vehicles. Lead formulations were selected based on solubility and stability measurements. For suspension development, suspensions in aqueous vehicles were prepared using a concentration of 250 mg/g micronized psilocin (PVP k17/Tween 80, HPβCD, Poloxamer 188). A suspension in a lipid-based vehicle was also prepared (corn oil/NMP/Kolliphor ELP). Based on solubility analyses, the lipid-based vehicle was taken forward for further testing.

Formulations Comprising Psilocybin

Vehicle components for the lead formulations were prepared as weight per volume in a volumetric flask. The suspensions (Formulations 1-3) were then prepared at a 1.6 g scale by weighing 144 mg of micronised psilocybin in a 7 mL glass vial and 1456 mg of vehicle, to give suspensions containing 90 mg/g psilocybin. The mixtures were stirred for approximately 3 hours at room temperature to achieve homogenous suspensions.

The formulations prepared are described in Table 6.

TABLE 6
Formulations comprising psilocybin

| | Composition |
|---|---|
| Formulation 1 | 90 mg/g micronised psilocybin in 0.5% |
| | polyvinylpyrrolidone (PVP) k17 + 0.1% Tween 80 in |
| | phosphate-buffered saline (PBS) |
| Formulation 2 | 90 mg/g micronised psilocybin in 10% HPβCD in |
| | PBS |
| Formulation 3 | 90 mg/g micronised psilocybin in 2% Poloxamer 188 |
| | in PBS |

Formulations Comprising Psilocin

Vehicle components were prepared as weight per volume in a volumetric flask for the solutions (Formulations 4 and 5) and as weight per weight for the suspension (Formulation 6). The solutions were then prepared at a 1 g scale by weighing 250 mg of psilocin in a 4 mL glass vial and 400 mg of vehicle. The mixtures were stirred whilst adding small aliquots of 5 M HCl by weight until pH 4 was achieved. Depending on the amount of HCl added to achieve pH 4, further vehicle was added to achieve a concentration of 250 mg/g psilocin. The suspension was prepared at a 1.2 g scale by weighing 300 mg of micronised psilocin in a 7 mL glass vial and 900 mg of vehicle, to give a suspension containing 250 mg/g psilocin. The mixture was stirred for approximately 3 hours at room temperature to achieve a homogenous suspension.

The formulations prepared are described in Table 7.

TABLE 7
Formulations comprising psilocin

| | Composition |
|---|---|
| Formulation 4 | 250 mg/g psilocin in 10% HPβCD (aq.) |
| Formulation 5 | 250 mg/g psilocin in 100% water |
| Formulation 6 | 250 mg/g micronised psilocin in 80% corn oil, 10% |
| | NMP, 10% Kolliphor ELP |

### Example 3

The following Example demonstrates the chemical and physical stability of the formulations of Example 2.

Analytical Methods

Equipment

The following equipment was used in Example 3.

| Instrument/Equipment | Series | Manufacturer |
|---|---|---|
| HPLC-UV (Psilocybin | 1220 Infinity II LC | Agilent |
| method) | | |
| HPLC-UV (Psilocin method) | 1100 | Agilent |
| pH-meter | SevenCompact | Mettler Toledo |
| pH electrode | In-Lab Micro | Mettler-Toledo |
| XRPD | D2 Phaser | Bruker |
| Heater/shaker | RS9000 | Barnstead STEM ™ |
| Osmometer | Osmomat ™ 3000 | Gonotec ® |

Psilocybin Analysis by HPLC-UV

The HPLC-UV method used for the analysis of psilocybin is detailed in Table 8. For each HPLC experiment, two independent standards (A and B) were prepared at a psilocybin (API) concentration of 0.1 mg/mL in 0.1% phosphoric acid in water (diluent). Approximately 2 mg of Psilocybin was weighed into a 20 mL volumetric pflask, followed by the addition of the diluent up to 20 mL and then sonicated until complete dissolution of API (approximately 10 minutes). The solutions were transferred to HPLC vials in preparation for HPLC analysis.

TABLE 8
Summary of the HPLC method used for psilocybin

| Column | YMC-Triart metal-free C18 | | |
|---|---|---|---|
| | (150 × 4.6 mm, 3 μm) | | |
| | S/N: 105YB10378 | | |
| | P/N: TA12503-1546PTP | | |
| Flow rate (mL/min) | 0.8 | | |
| Injection volume (μL) | 5 | | |
| Autosampler tray temperature (° C.) | 25 | | |
| Diluent | 0.1% phosphoric acid in water | | |
| Detection wavelength (nm) | 220 | | |
| Run time (min) | 47 | | |
| Standard concentration (mg/mL) | 0.1 | | |
| Mobile phase A | 0.1% phosphoric acid in water | | |
| Mobile phase B | 0.1% phosphoric acid in methanol | | |
| Column temperature (° C.) | 30 | | |
| | Gradient | | |
| | Time | Mobile | Mobile |
| Elution mode | (min) | phase A % | phase B % |
| | 0 | 95 | 5 |
| | 15 | 95 | 5 |
| | 18 | 85 | 15 |
| | 26 | 85 | 15 |
| | 35 | 70 | 30 |
| | 38 | 95 | 5 |
| | 47 | 95 | 5 |

Psilocin Analysis by HPLC-UV

The HPLC-UV method used for the analysis of psilocin is detailed in Table 9. For each HPLC experiment, two independent standards (A and B) were prepared at a psilocin (API) concentration of 0.1 mg/mL in 10 mM HCl (diluent). Approximately 2 mg of psilocin was weighed into a 20 mL volumetric flask, followed by the addition of the diluent up to 20 mL and then sonicated until complete dissolution of API (approximately 10 minutes). The solutions were transferred to HPLC vials in preparation for HPLC analysis.

TABLE 9
Summary of the HPLC method used for psilocin

| Column | Ascentis ® Express C18 | | |
|---|---|---|---|
| | (100 × 4.6 mm, 2.7 μm) | | |
| | Cat#53827-U | | |
| | Col: USPY010599 | | |
| Flow rate (mL/min) | 1 | | |
| Injection volume (μL) | 6 | | |
| Autosampler tray temperature (° C.) | 25 | | |
| Diluent | 10 mM HCl | | |
| Detection wavelength (nm) | 220 | | |
| Run time (min) | 25 | | |
| Standard concentration (mg/mL) | 0.1 | | |
| Mobile phase A | 0.05% TFA in water | | |
| Mobile phase B | Acetonitrile | | |
| Column temperature (° C.) | 30 | | |
| | Gradient | | |
| | Time | Mobile | Mobile |
| Elution mode | (min) | phase A % | phase B % |
| | 1 | 94 | 6 |
| | 10 | 82 | 18 |
| | 15 | 25 | 75 |
| | 20 | 25 | 75 |
| | 20.1 | 94 | 6 |
| | 25 | 94 | 6 |

X-Ray Powder Diffraction (XRPD)

X-ray powder diffraction (XRPD) analysis was carried out using a Bruker D2 Phaser powder diffractometer equipped with a LynxEye detector. The specimens were located at the centre of a silicon sample holder within a 5 mm pocket. The samples were continuously spun during data collection and scanned using a step size of 0.02° two theta (2θ) between 5° to 60° two theta, the detailed parameters are summarised in Table 10. The data was processed using Diffrac Plus EVA software (Version 3.1).

TABLE 10
Experimental parameters applied for XRPD analysis

| Parameter | Condition |
|---|---|
| Instrument | Bruker D2 Phaser |
| Scan mode | Continuous PSD fast |
| Source | Copper |
| Wavelength | Kα = 1.54060 nm |
| 2 Theta Range (Start/Stop) | 5-60º 2θ |
| Detector | Lynx Eye |
| Sample movement | Spinning |
| Generator/voltage | 30 kV/10 mA |
| 2 Theta Step Size | 0.02º |
| Time/Step (Dwell) | 0.2 seconds |

Particle Size Distribution (PSD) Analysis

Particle size analysis was carried out, in single analysis, by laser diffraction using a Sympatec Helos particle size analyser, equipped with a RODOS dry dispersion unit and ASPIROS feeder unit. The experimental parameters used are listed in Table 11. The lens and primary pressures were varied for each compound tested.

TABLE 11
Experimental parameters applied for PSD analysis

| Parameter | Condition |
|---|---|
| Instrument | Sympatec Helos |
| Software | Sympatec Windox 5.0 |
| Dispersing accessory | Aspiros |
| Trigger conditions | Time base: 1.0 ms |
| | Measurement: standard mode |
| | Start 0.000 s opt. concentration ≥1.0% |
| | Stop after 5 s opt. concentration ≤1.0% |
| | Trigger time out 60 s |

Stability Testing of Formulations Comprising Psilocybin

Formulations 1-3 were stored in closed glass vials in a 25° C. incubator for analysis at each time-point. At the following time-points: t=0, 1, 4 and 7 days, the chemical and physical stability of the formulations was assessed visually, by measuring the pH and osmolality, by assaying for homogeneity and dissolved fraction, and characterising the undissolved content by XRPD.

To assay the suspensions for homogeneity, 10 μL of the suspension from the top and bottom sections was weighed into separate 10 mL volumetric flasks, in duplicate, and the volume was filled with 0.1% phosphoric acid in water. These were vortexed for approximately 20 seconds and then transferred to HPLC vials for assay and purity quantification.

To assay the suspensions for dissolved fraction, 100 μL of the suspension was centrifuged in a 0.5 mL Eppendorf for 10 minutes at 12,500 rpm. 10 μL of the supernatant was then diluted with 990 μL 0.1% phosphoric acid in water directly in an HPLC vial (v/v) and vortexed for approximately 10 seconds. The pellet left in the Eppendorf was used for XRPD characterisation.

Results

The results are shown in Table 12.

TABLE 12
Stability results for Formulations 1-3

| | | | | | | Bottom | |
|---|---|---|---|---|---|---|---|
| | Time- | | | | Top section | section assay | |
| | point/ | | | Dissolved | assay (%)/ | (%)/ | |
| | storage | | Osmolality | fraction | concentration | concentration | Purity |
| Formulation | condition | pH | (mOsm/kg) | (mg/g) | (mg/g) | (mg/g) | (%) |
| 1 | t = 0 | 5.3 | 314 | 8.4 ± 0.09 | 94.1 ± 1.8/ | 97.1 ± 10.1/ | 99.4 |
| | | | | | 84.7 ± 1.6 | 87.4 ± 9.1 | |
| | t = 24 h/ | 5.5 | 355 | 8.3 ± 0.04 | 93.8 ± 0.7/ | 95.4 ± 0.7/ | 99.4 |
| | 25° C. | | | | 84.4 ± 0.7 | 85.8 ± 0.6 | |
| | t = 4 d/ | 5.5 | 336 | 8.6 ± 0.04 | 91.1 ± 0.9/ | 91.6 ± 1.0/ | 99.4 |
| | 25° C. | | | | 82.0 ± 0.8 | 82.4 ± 0.9 | |
| | t = 7 d/ | 5.3 | 455 | 9.3 ± 0.07 | 102.5 ± 2.8/ | 101.1 ± 2.3/ | 99.4 |
| | 25° C. | | | | 92.3 ± 2.5 | 91.0 ± 2.0 | |
| 2 | t = 0 | 5.2 | 408 | 9.2 ± 0.1 | 98.0 ± 1.0/ | 97.1 ± 4.2/ | 99.4 |
| | | | | | 87.9 ± 0.9 | 87.0 ± 3.7 | |
| | t = 24 h/ | 5.4 | 428 | 9.3 ± 0.3 | 94.8 ± 0.6/ | 93.6 ± 0.8/ | 99.4 |
| | 25° C. | | | | 85.0 ± 0.5 | 83.9 ± 0.7 | |
| | t = 4 d/ | 5.4 | 474 | 9.5 ± 0.3 | 93.3 ± 4.4/ | 90.1 ± 0.1/ | 99.4 |
| | 25° C. | | | | 83.6 ± 3.9 | 80.8 ± 0.1 | |
| | t = 7 d/ | 5.2 | 511 | 10.6 ± 0.4 | 95.1 ± 0.1/ | 105.3 ± 0.2/ | 99.4 |
| | 25° C. | | | | 85.2 ± 0.1 | 94.4 ± 0.1 | |
| 3 | t = 0 | 5.2 | 325 | 8.5 ± 0.08 | 96.1 ± 1.0/ | 86.4 ± 3.0/ | 99.4 |
| | | | | | 86.8 ± 0.9 | 78.0 ± 2.7 | |
| | t = 24 h/ | 5.4 | 388 | 8.6 ± 0.04 | 86.9 ± 0.2/ | 90.3 ± 0.1/ | 99.4 |
| | 25 ° C. | | | | 78.5 ± 0.2 | 81.6 ± 0.1 | |
| | t = 4 d/ | 5.4 | 351 | 8.6 ± 0.03 | 99.7 ± 7.0/ | 85.6 ± 0.05/ | 99.4 |
| | 25° C. | | | | 90.1 ± 6.3 | 86.3 ± 0.05 | |
| | t = 7 d/ | 5.4 | 370 | 9.5 ± 0.1 | 86.6 ± 3.7/ | 82.6 ± 1.7/ | 99.3 |
| | 25° C. | | | | 78.2 ± 3.3 | 74.6 ± 1.6 | |

In conclusion, Formulations 1-3 were physically and chemically stable for at least 7 days at 25° C. The percentage peak area for all formulations after 7 days is comparable to t=0 (i.e. no degradation detected by HPLC). The pH and osmolality values were also comparable. XRPD analysis of all 3 suspensions showed conversion of psilocybin to the trihydrate form from t=0 and no changes for at least 7 days.

Stability Testing of Formulations Comprising Psilocin

Formulations 4-6 were stored in closed glass vials at 2-8° C. for stability testing. At the following time-points: t=0, 1, 5 and 7 days, the chemical and physical stability of the solutions was assessed visually, by measuring the pH and osmolality, and by assay. The stability of the suspension was assessed visually, by assaying for homogeneity and dissolved fraction, and characterising the undissolved content by XRPD at the same time-points.

To assay the solutions, 50 μL of the solution was first centrifuged at 12,500 rpm for 10 minutes. Next, 10 μL of the supernatant was weighed into a 25 mL volumetric flask, in duplicate, and the volume was filled with 10 mM HCl. The volumetric flasks were then vortexed for approximately 30 seconds and then transferred to HPLC vials for assay and purity quantification.

To assay the suspensions for homogeneity, 10 μL of the suspension from the top and bottom sections was weighed into separate 25 mL volumetric flasks, in duplicate, and the volume was filled DCM. These were vortexed for approximately 30 seconds and then transferred to HPLC vials for assay and purity quantification.

To assay the suspensions for dissolved fraction, 100 μL of the suspension was centrifuged in a 0.5 mL Eppendorf for 10 minutes at 12,500 rpm. 10 μL of the supernatant was then diluted with 190 μL of DCM directly in an HPLC vial (v/v) and vortexed for approximately 10 seconds. The pellet left in the Eppendorf was used for XRPD characterisation.

Results

The results are shown in Tables 13 and 14.

TABLE 13
Stability results for formulations 4 and 5

| | Time- | | | | Measured | |
|---|---|---|---|---|---|---|
| | point/ | | | | concen- | |
| Formu- | storage | | Osmolality | Assay | tration | Purity |
| lation | condition | pH | (mOsm/kg) | (%) | (mg/g) | (%) |
| 4 | t = 0 | 4.2 | 1521 | 110.9 ± | 278.2 ± 5.9 | 99.2 |
| | | | | 2.3 | | |
| | t = 24 h/ | 3.9 | 1525 | 116.4 ± | 292.0 ± 1.2 | 99.2 |
| | 2-8° C. | | | 0.5 | | |
| | t = 5 d/ | 3.7 | 1526 | 125.4 ± | 314.5 ± 31.4 | 99.1 |
| | 2-8° C. | | | 12.5 | | |
| | t = 7 d/ | 3.7 | 1532 | 112.5 ± | 282.2 ± 19.4 | 99.2 |
| | 2-8° C. | | | 7.7 | | |
| 5 | t = 0 | 4.2 | 1211 | 114.4 ± | 287.3 ± 8.6 | 99.2 |
| | | | | 3.4 | | |
| | t = 24 h/ | 4.3 | 1212 | 103.3 ± | 259.3 ± 11.9 | 99.2 |
| | 2-8° C. | | | 4.7 | | |
| | t = 5 d/ | 4.1 | 1205 | 108.9 ± | 273.5 ± 5.5 | 99.2 |
| | 2-8° C. | | | 2.2 | | |
| | t = 7 d/ | 4.2 | 1207 | 105.9 | 265.9 | 99.2 |
| | 2-8° C. | | | | | |

TABLE 14
Stability results for formulation 6

| | | | | Bottom | |
|---|---|---|---|---|---|
| | Time- | | Top section | section assay | |
| | point/ | Dissolved | assay (%)/ | (%)/ | |
| For- | storage | fraction | concentration | concentration | Purity |
| mulation | condition | (mg/g) | (mg/g) | (mg/g) | (%) |
| 6 | t = 24 h/ | 6.4 ± 0.1 | 101.3 ± 4.0/ | 105.0 ± 5.1/ | 98.8 |
| | 2-8° C. | | 251.0 ± 9.8 | 260.2 ± 12.6 | |
| | t = 5 d/ | 7.0 ± 0.1 | 98.3 ± 6.4/ | 101.9 ± 3.0/ | 98.8 |
| | 2-8° C. | | 243.4 ± 15.9 | 252.5 ± 7.5 | |
| | t = 7 d/ | 6.6 ± 0.0003 | 105.1 ± 12.6/ | 104.5 ± 3.5/ | 98.9 |
| | 2-8° C. | | 260.4 ± 31.3 | 258.8 ± 8.7 | |

In conclusion, the solutions and suspension were chemically and physically stable upon storage at 2-8° C. for at least 7 days. For the solutions, there was no reduction over time in the percentage peak area as measured by HPLC (i.e. no degradation). It is thought that adjusting the pH of the formulations to pH 4 prevents degradation of psilocin.

For the suspension, due to an HPLC error, the t=0 results were not obtainable. The HPLC results from the 1- and 7-days' time-points show that the suspension is homogenous with no reduction in assay or percentage peak area, indicating no degradation detected by HPLC over the 7-day period. XRPD analysis showed no change in physical form for at least 7 days.

### CONCLUSION

It has been shown that the formulations described herein are advantageously suitable for administration by subcutaneous injection, and show good chemical and physical stability.

1. A method of treating or preventing a disease or condition in a patient, the method comprising administering a therapeutically effective amount of a psychedelic compound to the patient by subcutaneous injection,
wherein the psychedelic compound is psilocybin or psilocin, or a pharmaceutically acceptable salt thereof.

2. A method according to claim 1, wherein the psychedelic compound is psilocybin or a pharmaceutically acceptable salt thereof.

3. A method according to claim 1, wherein the psychedelic compound is psilocin or a pharmaceutically acceptable salt thereof.

4. The method according to any one of the preceding claims, wherein the therapeutically effective amount of the psychedelic compound is from about 0.5 mg to about 25 mg, from about 1 mg to about 24 mg, from about 1.5 mg to about 23 mg, from about 2 mg to about 22 mg, from about 2.5 mg to about 21 mg, from about 3 mg to about 20 mg, from about 3.5 mg to about 19 mg, from about 4 mg to about 18 mg, from about 4.5 mg to about 17 mg, from about 5 mg to about 16 mg, from about 5.5 mg to about 15 mg, from about 6 mg to about 14 mg, from about 6.5 mg to about 13 mg, or from about 7 mg to about 12 mg.

5. The method according to any one of the preceding claims, wherein the therapeutically effective amount of the psychedelic compound is from about 3.5 mg to about 4.5 mg, from about 4.5 mg to about 5.5 mg, from about 5.5 mg to about 6.5 mg, from about 6.5 mg to about 7.5 mg, from about 7.5 mg to about 8.5 mg, from about 8.5 mg to about 9.5 mg, from about 9.5 mg to about 10.5 mg, from about 10.5 mg to about 11.5 mg, from about 11.5 mg to about 12.5 mg, from about 12.5 mg to about 13.5 mg, from about 13.5 mg to about 14.5 mg, from about 14.5 mg to about 15.5 mg, from about 15.5 mg to about 16.5 mg, or from about 16.5 mg to about 17.5 mg.

6. The method according to any one of the preceding claims, wherein administering the therapeutically effective amount of the psychedelic compound to the patient by subcutaneous injection achieves a maximum blood plasma concentration (Cₘₐₓ) of psilocin in the patient which is at least about 3 ng/mL, at least about 4 ng/mL, at least about 5 ng/mL, at least about 6 ng/mL, at least about 7 ng/mL, at least about 8 ng/mL, at least about 9 ng/mL, or at least about 10 ng/mL.

7. The method according to any one of the preceding claims, wherein administering the therapeutically effective amount of the psychedelic compound to the patient by subcutaneous injection achieves a maximum blood plasma concentration (Cₘₐₓ) of psilocin in the patient which is no greater than about 20 ng/mL, no greater than about 18 ng/mL, no greater than about 16 ng/mL, no greater than about 14 ng/mL, no greater than about 12 ng/mL, or no greater than about 10 ng/mL.

8. The method according to any one of the preceding claims, wherein administering the therapeutically effective amount of the psychedelic compound to the patient by subcutaneous injection achieves a maximum blood plasma concentration (Cₘₐₓ) of psilocin in the patient which is from about 3 ng/mL to about 4 ng/mL, from about 4 ng/mL to about 5 ng/mL, from about 5 ng/mL to about 6 ng/mL, from about 6 ng/mL to about 7 ng/mL, from about 7 ng/mL to about 8 ng/mL, from about 8 ng/mL to about 9 ng/mL, from about 9 ng/mL to about 10 ng/mL, from about 10 ng/mL to about 11 ng/mL, from about 11 ng/mL to about 12 ng/mL, from about 12 ng/mL to about 13 ng/mL, from about 13 ng/mL to about 14 ng/mL, or from about 14 ng/mL to about 15 ng/mL.

9. The method according to any one of the preceding claims, wherein the method comprises administering a pharmaceutical composition comprising a therapeutically effective amount of the psychedelic compound to a patient by subcutaneous injection,
which pharmaceutical composition is a formulation selected from a solution, suspension, emulsion, gel, liposome poorly soluble salt formulation, oily depot, viscous depot, protein binding system, lipidic system, polymer system, particulate system, or an in-situ gelling system.

10. The method according to claim 9, wherein the pharmaceutical composition is a formulation which is a solution or a formulation which is a suspension.

11. The method according to claim 9 or claim 10, wherein the formulation comprises the psychedelic compound at a concentration of at least about 50 mg/g.

12. The method according to claim 11, wherein the formulation comprises the psychedelic compound at a concentration of at least about 70 mg/g.

13. The method according to claim 12, wherein the formulation comprises the psychedelic compound at a concentration of at least about 90 mg/g.

14. The method according to claim 13, wherein the formulation comprises the psychedelic compound at a concentration of at least about 200 mg/g.

15. The method according to any one of claims 9 to 14, wherein the formulation is a suspension comprising particles of the psychedelic compound and the particles of the psychedelic compound have a D₅₀ of less than about 3 μm.

16. The method according to claim 15, wherein the particles of the psychedelic compound in the formulation have a D₅₀ of less than about 1.5 μm.

17. The method according to any one of claims 9 to 16, wherein the formulation is a suspension comprising particles of the psychedelic compound and the particles of the psychedelic compound in the formulation have a D₉₀ of from 2.0 to 4.0 μm.

18. The method according to any one of claims 9 to 17, wherein the formulation is a suspension comprising particles of the psychedelic compound, polyvinylpyrrolidone, polyoxyethylene (80) sorbitan monooleate, and phosphate-buffered saline, and wherein the psychedelic compound is psilocybin.

19. The method according to any one of claims 9 to 17, wherein the formulation is a suspension comprising particles of the psychedelic compound, 2-hydroxypropyl-β-cyclodextrin, and phosphate-buffered saline, and wherein the psychedelic compound is psilocybin.

20. The method according to any one of claims 9 to 17, wherein the formulation is a suspension comprising particles of the psychedelic compound, polyethylene-polypropylene glycol, and phosphate-buffered saline, and wherein the psychedelic compound is psilocybin.

21. The method according to any one of claims 9 to 14, wherein the formulation is a solution comprising the psychedelic compound, 2-hydroxypropyl-β-cyclodextrin, and water, and wherein the psychedelic compound is psilocin.

22. The method according to any one of claims 9 to 14, wherein the formulation is a solution comprising the psychedelic compound and water, and wherein the psychedelic compound is psilocin.

23. The method according to any one of claims 9 to 17, wherein the formulation is a suspension comprising particles of the psychedelic compound, corn oil, N-methylpyrrolidone, and polyoxyl-35-castor oil, wherein the psychedelic compound is psilocin.

24. The method according to any one of the preceding claims, wherein administering the therapeutically effective amount of the psychedelic compound to the patient by subcutaneous injection comprises administering:
a first dose of the psychedelic compound with a first absorption half-life of the psychedelic compound of (t_{firstdose})_{1/2}; and
a second dose of the psychedelic compound with a second absorption half-life of the psychedelic compound of (t_{seconddose})_{1/2};
wherein (t_{seconddose})_{1/2}>(t_{firstdose})_{1/2}.

25. The method according to claim 24, wherein (t_{seconddose})_{1/2}>n (t_{firstdose})_{1/2}; and wherein n=1, 2, 3, 4, 5, 6, 7, 8, 9, 10, 11, 12, 13, 14, 15, 16, 17, 18, 19, or 20.

26. The method according to claim 24 or claim 25, wherein the first dose is from about 0.1 mg to about 10 mg, from about 0.2 mg to about 9 mg, from about 0.3 mg to about 8 mg, from about 0.4 mg to about 7 mg, from about 0.5 mg to about 6 mg, from about 0.6 mg to about 5 mg, from about 0.7 mg to about 4 mg, from about 0.8 mg to about 3 mg, from about 0.9 mg to about 2 mg, or from about 1 mg to about 1.5 mg.

27. The method according to any one of claims 24 to 26, wherein the first dose is from about 0.1 mg to about 0.3 mg, from about 0.3 mg to about 0.5 mg, from about 0.5 mg to about 0.7 mg, from about 0.7 mg to about 0.9 mg, from about 0.9 mg to about 1.1 mg, from about 1.1 mg to about 1.3 mg, from about 1.3 mg to about 1.5 mg, from about 1.5 mg to about 1.7 mg, or from about 1.7 mg to about 1.9 mg.

28. The method according to any one of claims 24 to 27, wherein the first dose is less than or equal to 50%, less than or equal to 40%, less than or equal to 30%, less than or equal to 20%, or less than or equal to 10% of the second dose.

29. The method according to any one of claims 24 to 28, wherein (t_{firstdose})_{1/2} is from about 0.5 minutes to about 15 minutes, from about 1 minute to about 8 minutes, from about 2 minutes to about 7 minutes, from about 3 minutes to about 6 minutes, or from about 4 minutes to about 5 minutes.

30. The method according to any one of claims 24 to 29, wherein (t_{seconddose})_{1/2} is from about 10 minutes to about 200 minutes, from about 20 minutes to about 170 minutes, from about 30 minutes to about 140 minutes, from about 40 minutes to about 110 minutes, or from about 50 minutes to about 100 minutes.

31. The method according to any one of claims 24 to 30, wherein the first and second doses are administered as a single subcutaneous injection.

32. The method according to any one of claims 24 to 31, wherein the first dose of the psychedelic compound is formulated as a first pharmaceutical composition which is a first formulation selected from a solution, suspension, emulsion, gel, liposome poorly soluble salt formulation, oily depot, viscous depot, protein binding system, lipidic system, polymer system, particulate system, or an in-situ gelling system.

33. The method according to claim 32, wherein the first formulation is a formulation as defined in any one of claims 10 to 23.

34. The method according to any one of claims 24 to 33, wherein the second dose of the psychedelic compound is formulated as a second pharmaceutical composition which is a second formulation selected from a solution, suspension, emulsion, gel, liposome poorly soluble salt formulation, oily depot, viscous depot, protein binding system, lipidic system, polymer system, particulate system, or an in-situ gelling system.

35. The method according to claim 34, wherein the second formulation is a formulation as defined in any one of claims 10 to 23.

36. The method according to any one of claims 24 to 35, wherein administering the therapeutically effective amount of the psychedelic compound to the patient by subcutaneous injection further comprises administering a third dose of the psychedelic compound with a third absorption half-life of the psychedelic compound of (t_{thirddose})_{1/2}.

37. The method according to any one of the preceding claims, wherein the disease or condition is selected from a psychological, neurological and central nervous system disorder.

38. The method according to any one of the preceding claims, wherein the disease or condition is selected from depression, anxiety, death anxiety, demoralization, hopelessness, adjustment disorders, suicidal ideation and desire for hastened death.

39. The method according to any one of claims 1 to 37, wherein the disease or condition is selected from cocaine-related disorders, opioid-related disorders and stimulant-related disorders.

40. A psychedelic compound for use in a method of treating or preventing a disease or condition in a patient, wherein:
the psychedelic compound is psilocybin or psilocin, or a pharmaceutically acceptable salt thereof; and
the method comprises administering a therapeutically effective amount of a psychedelic compound to the patient by subcutaneous injection.

41. The psychedelic compound for use according to claim 40, wherein:
administering the psychedelic compound by subcutaneous injection is as further defined in any one of claims 2 to 36.

42. The psychedelic compound for use according to claim 40 or claim 41, wherein the psychedelic compound is for use in the treatment of a disease or condition selected from a psychological, neurological and central nervous system disorder, and preferably selected from depression, anxiety, death anxiety, demoralization, hopelessness, adjustment disorders, suicidal ideation and desire for hastened death.

43. The psychedelic compound for use according to claim 40 or claim 41, wherein the psychedelic compound is for use in the treatment of a disease or condition selected from cocaine-related disorders, opioid-related disorders and stimulant-related disorders.

44. Use of a psychedelic compound in the manufacture of a medicament for use in a method of treating or preventing of a disease or condition in a patient, wherein:
the psychedelic compound is psilocybin or psilocin, or a pharmaceutically acceptable salt thereof; and
the method comprises administering a therapeutically effective amount of a psychedelic compound to the patient by subcutaneous injection.

45. A formulation suitable for administration by subcutaneous injection comprising a psychedelic compound which is psilocybin or a pharmaceutically acceptable salt thereof, wherein the formulation is a suspension and the formulation comprises the psychedelic compound at a concentration of at least about 70 mg/g.

46. A formulation suitable for administration by subcutaneous injection comprising a psychedelic compound which is psilocin or a pharmaceutically acceptable salt thereof, wherein the formulation is a suspension or a solution and the formulation comprises psilocin at a concentration of at least about 200 mg/g.

47. The formulation according to claim 45 or 46, wherein the formulation is a suspension comprising particles of the psychedelic compound and the particles of the psychedelic compound have a D₅₀ of less than about 3 μm.

48. The formulation according to claim 47, wherein the formulation is a suspension comprising particles of the psychedelic compound and the particles of the psychedelic compound have a D₅₀ of less than about 1.5 μm.

49. The formulation according to any one of claims 45 to 47, wherein the formulation is a suspension comprising particles of the psychedelic compound and the particles of the psychedelic compound have a D₉₀ of from 2.0 to 4.0 μm.

50. The formulation according to any one of claims 45 to 49, wherein the formulation further comprises at least one pharmaceutically acceptable diluent, and optionally further comprises at least one pharmaceutically acceptable buffer, solubiliser, polymer, and/or surfactant.

51. The formulation according to claim 50, wherein the formulation is a suspension comprising polyvinylpyrrolidone, polyoxyethylene (80) sorbitan monooleate, and phosphate-buffered saline, and wherein the psychedelic compound is psilocybin.

52. The formulation according to claim 51, wherein the formulation comprises polyvinylpyrrolidone at a concentration of from 0.1 to 1.0% w/v, polyoxyethylene (80) sorbitan monooleate at a concentration of from 0.01 to 0.50% w/v, and phosphate-buffered saline at a concentration of at least 98.5% w/v.

53. The formulation according to claim 50, wherein the formulation is a suspension comprising 2-hydroxypropyl-β-cyclodextrin and phosphate-buffered saline, and wherein the psychedelic compound is psilocybin.

54. The formulation according to claim 53, wherein the formulation comprises 2-hydroxypropyl-β-cyclodextrin at a concentration of from 5 to 15% w/v, and phosphate-buffered saline at a concentration of from 85 to 95% w/v.

55. The formulation according to claim 50, wherein the formulation is a suspension comprising polyethylene-polypropylene glycol and phosphate-buffered saline, and wherein the psychedelic compound is psilocybin.

56. The formulation according to claim 55, wherein the formulation comprises polyethylene-polypropylene glycol at a concentration of from 1 to 5% w/v, and phosphate-buffered saline at a concentration of from 95 to 99% w/v.

57. The formulation according to claim 50, wherein the formulation is a solution comprising 2-hydroxypropyl-β-cyclodextrin and water, and wherein the psychedelic compound is psilocin.

58. The formulation according to claim 57, wherein the formulation comprises 2-hydroxypropyl-β-cyclodextrin at a concentration of from 5 to 15% w/v, and water at a concentration of from 85 to 95% w/v.

59. The formulation according to claim 50, wherein the formulation is a solution comprising water, and wherein the psychedelic compound is psilocin.

60. The formulation according to claim 59, wherein the formulation comprises 100% w/v water.

61. The formulation according to claim 50, wherein the formulation is a suspension comprising corn oil, N-methylpyrrolidone and polyoxyl-35-castor oil, and wherein the psychedelic compound is psilocin.

62. The formulation according to claim 61, wherein the formulation comprises corn oil at a concentration of from 70 to 90% w/w, N-methylpyrrolidone at a concentration of from 5 to 15% w/w, and polyoxyl-35-castor oil at a concentration of from 5 to 15% w/w.

63. A formulation according to any one of claims 45 to 62, wherein the formulation comprises psilocybin and is stable for 7 days at 25° C.

64. A formulation according to any one of claims 45 to 62, wherein the formulation comprises psilocin and is stable for 7 days at 2-8° C.

65. A kit comprising:
one or more formulations suitable for administration by subcutaneous injection, which one or more formulations comprise a psychedelic compound which is psilocybin or psilocin, or a pharmaceutically acceptable salt thereof; and
instructions for use of the one or more formulations in a method as defined in any one of claims 1 to 39.

66. The kit of claim 65, wherein each of the one or more formulations are as defined in any one of claims 45 to 64.
